(19) United States
(12) Patent Application Publication
(10) Pub. No.: US 2026/0248697 A1
(43) Pub. Date: Aug. 27, 2026

(54) COMPOSITION COMPRISING A PEROXYGENATED SALT, AN ASSOCIATIVE POLYMER, A NON-ASSOCIATIVE POLYSACCHARIDE AND A HYDROCARBON WITH A MELTING POINT GREATER THAN 25°C

(71) Applicant: L'OREAL, Paris (FR)

(72) Inventors: Frédéric SIMONET, CHEVILLY-LARUE (FR); Hugo DORIE, SAINT-OUEN (FR); Benoît BOCHE, SAINT-OUEN (FR)

(21) Appl. No.: 18/879,141

(22) PCT Filed: Jun. 28, 2023
(86) PCT No.: PCT/EP2023/067720

§ 371 Date: Dec. 26, 2024

(30) Foreign Application Priority Data
Jun. 29, 2022 (FR) FR2206508

(51) Int. Cl. A61K 8/23; A61K 8/22; A61K 8/25; A61K 8/31; A61K 8/73; A61K 8/81; A61Q 5/08

(52) U.S. Cl. CPC A61K 8/23; A61K 8/22; A61K 8/25; A61K 8/31; A61K 8/731; A61K 8/8111; A61K 8/8147; A61K 8/8152; A61Q 5/08; A61K 2800/87; A61K 2800/882

(57) ABSTRACT

The invention relates to a composition for lightening keratin fibres, and in particular human keratin fibres such as the hair, comprising one or more peroxygenated salts, one or more associative polymer(s), one or more non-associative polysaccharide(s) and one or more hydrocarbon(s) with a melting point of greater than 25° C.

The invention also relates to a process for lightening keratin fibres, preferably human keratin fibres, particularly the hair, comprising the application of the composition to said keratin fibres.

A subject of the present invention is a composition for lightening keratin fibres, and in particular human keratin fibres such as the hair, comprising one or more peroxygenated salts, one or more associative polymer(s), one or more non-associative polysaccharide(s) and one or more hydrocarbon(s) with a melting point of greater than 25° C.

The invention also relates to the process for lightening human keratin fibres using such a composition.

In the field of hair lightening, tone depth is generally used to characterize the degree or level of lightening. The concept of “tone” is based on the classification of natural shades, with one tone separating each shade from the shade immediately following or preceding it. This definition and the classification of natural shades are well known to hairstyling professionals and are published in the book Sciences des traitements capillaires [The Science of Hair Care] by Charles Zviak, 1988, published by Masson, pages 215 and 278.

The tone depths range from 1 (black) to 10 (lightest blond), with one unit corresponding to one tone; the higher the number, the lighter the shade.

Lightening thus makes it possible to provide a lighter tone depth than the initial natural tone depth of the head of hair.

The processes used to lighten the hair generally consist in using an aqueous composition comprising at least one oxidizing agent, under alkaline pH conditions in the vast majority of cases.

The role of this oxidizing agent is to degrade the melanin of the hair, which, depending on the nature of the oxidizing agent present, leads to more or less pronounced lightening of the fibres. Thus, for relatively mild lightening, the oxidizing agent is generally hydrogen peroxide. When more pronounced lightening is desired, particularly lightening by at least 5 tones, use is usually made of peroxygenated salts, for instance persulfates, in the presence of hydrogen peroxide. These peroxygenated salts are contained in compositions which, at the time of use, are mixed with an aqueous composition comprising hydrogen peroxide.

In order to adjust the pH of the compositions to an alkaline pH to enable activation of the oxidizing agent, use is made of an alkaline agent. This alkaline agent also causes swelling of the keratin fibre, with opening of the scales, which promotes the penetration of the oxidizing agent into the fibre, and thus increases the efficacy of the reaction.

However, the use of alkaline agents and peroxygenated salts may have an adverse effect on the quality of the hair. The essential causes of this adverse effect on the quality of the hair are a decrease in its cosmetic properties, such as its sheen, and degradation of its mechanical properties, more particularly degradation of its mechanical strength, which may also be reflected by an increase in its porosity. The hair is weakened and may become brittle during subsequent treatments such as blow-drying. Increased frizz, which is not particularly attractive, is also observed.

Lightening dark hair is therefore particularly tricky because it requires the use of a significant amount of peroxygenated salts if it is desired to greatly lighten the hair, and this may make the hair brittle.

In addition, lightening compositions applied to extremely curly hair tend to modify the shape of the curls, which are generally less well-defined.

Moreover, compositions containing peroxygenated salts are generally in powder form. Since, however, pulverulent compositions have the disadvantage of producing dust when they are handled, transported and stored, compositions in paste form have been proposed. Pulverulent compositions are thus dispersed in a thickened organic inert liquid support which provides a solution to the problems of volatility.

However, the use of compositions in paste form causes new issues.

These pastes are generally anhydrous with a compact, hard texture. As a result, the mixing of the paste and the hydrogen peroxide composition is far from simple. This is reflected not only in a longer mixing time but also in difficulties obtaining a homogeneous and stable mixture.

Moreover, the lightening compositions obtained can be difficult to spread uniformly over a whole head of hair, in particular curly or very curly heads of hair, which can lead to undesired patchy lightening performance.

Thus, one of the objectives of the present invention is to propose compositions for lightening keratin materials, preferably human keratin fibres such as the hair, which do not have the disadvantages mentioned above, i.e. which are capable of providing very good lightening performance without detrimentally affecting the cosmetic properties of the hair and while having very good usage qualities.

These aims, and others, are achieved by the present invention, one subject of which is thus a composition comprising:
one or more peroxygenated salts;
one or more associative polymers;
one or more non-associative polysaccharides; and
one or more hydrocarbon(s) with a melting point of greater than 25° C.

According to a preferred embodiment, the composition according to the invention is a composition for lightening keratin fibres, preferably human keratin fibres, preferably hair.

The invention also relates to a lightening process implementing said composition, to the use of the composition for lightening keratin fibres, and in particular the hair, and also to a multi-compartment device suitable for implementing said lightening process.

The composition according to the invention leads to a significant level of lightening, extending up to 9 tones, without any major adverse effect on the cosmetic properties of the hair, and has improved usage qualities.

In particular, the composition according to the invention is quickly and easily mixed with an aqueous composition of hydrogen peroxide in order to obtain a homogeneous and stable mixture. The mixture is easy to apply to the hair. Its presentation form, as a smooth cream, makes it possible to prevent running during application, while spreading easily throughout the head of hair, even in the case of extremely curly hair. In addition, the mixture does not dry during the leave-on time, enabling the active ingredients to be optimally available throughout the whole leave-on time. Moreover, the mixture rinses out easily.

The cosmetic properties of hair treated with the composition according to the invention are not significantly adversely affected, particularly in terms of softness and disentangling. The composition makes it possible in particular to condition the hair and to limit hair breakage during disentangling of the hair, particularly breakage of extremely curly hair. When it is applied to extremely curly hair, it also makes it possible to maintain the shape of the curls, affording them good definition. The composition also makes it possible to have good frizz control.

Other subjects, features, aspects and advantages of the invention will become even more apparent on reading the description and the examples that follow.

In the text hereinbelow, unless otherwise indicated, the limits of a range of values are included in that range, particularly in the expressions “between” and “ranging from . . . to . . . ”.

Moreover, the expression “at least one” used in the present description is equivalent to the expression “one or more”.

### Peroxygenated Salts

The composition according to the invention comprises one or more peroxygenated salts.

Preferably, the peroxygenated salts are chosen from persulfates; perborates; peracids and/or salts thereof; alkali metal, alkaline-earth metal or ammonium percarbonates; magnesium peroxide; and mixtures thereof.

More preferentially, the composition according to the present invention comprises at least one persulfate.

Persulfates, also known as peroxysulfates, correspond, for the purposes of the invention, to SO₅²⁻ anions (peroxomonosulfate anion) or S₂O₈²⁻ anions (peroxodisulfate anion) or to compounds comprising at least one of these anions.

Preferably, the persulfates according to the invention are chosen from peroxodisulfates.

According to a preferred embodiment of the invention, the composition according to the invention comprises at least one peroxygenated salt chosen from persulfates; preferably from alkali metal persulfates, alkaline-earth metal persulfates, ammonium persulfates, and mixtures thereof; more preferentially from (bis)tetrabutylammonium persulfate, barium persulfate, magnesium persulfate, calcium persulfate, sodium persulfate, potassium persulfate, ammonium persulfate, and mixtures thereof; even more preferentially from sodium persulfate, potassium persulfate, ammonium persulfate, and mixtures thereof; even better still from potassium persulfate, ammonium persulfate and mixtures thereof.

Preferably, the total content of peroxygenated salt(s) present in the composition according to the invention ranges from 1% to 60% by weight, more preferentially from 5% to 55% by weight, even more preferentially from 10% to 50% by weight, even better still from 20% to 45% by weight, indeed even from 30% to 40% by weight, relative to the total weight of the composition.

Preferably, the total content of persulfate(s) present in the composition according to the invention ranges from 1% to 60% by weight, more preferentially from 5% to 55% by weight, even more preferentially from 10% to 50% by weight, even better still from 20% to 45% by weight, indeed even from 30% to 40% by weight, relative to the total weight of the composition.

### Associative Polymers

The composition according to the invention also comprises at least one associative polymer.

It is recalled that “associative polymers”, preferably amphiphilic, are polymers that are capable, in an aqueous medium, of reversibly associating with one another or with other molecules.

Their chemical structure more particularly comprises at least one hydrophilic zone or group and at least one hydrophobic zone or group.

“Hydrophobic zone or group” means a radical or polymer with a saturated or unsaturated, linear or branched hydrocarbon-based chain, comprising at least 10 carbon atoms, preferably from 10 to 30 carbon atoms, in particular from 12 to 30 carbon atoms and more preferentially from 18 to 30 carbon atoms.

Preferentially, the hydrocarbon-based group originates from a monofunctional compound. By way of example, the hydrophobic group may be derived from a fatty alcohol such as stearyl alcohol, dodecyl alcohol or decyl alcohol. It may also denote a hydrocarbon-based polymer, for instance polybutadiene.

The associative polymer may be of anionic, cationic, amphoteric or nonionic type.

Among the associative polymers of anionic type, mention may be made of:
(a) those comprising at least one hydrophilic unit and at least one fatty-chain allyl ether unit, more particularly those whose hydrophilic unit is constituted by an ethylenically unsaturated anionic monomer, more particularly still by a vinylcarboxylic acid and most particularly by an acrylic acid or a methacrylic acid or mixtures thereof.

Among these anionic associative polymers, those that are particularly preferred according to the invention are polymers formed from 20% to 60% by weight of acrylic acid and/or of methacrylic acid, from 5% to 60% by weight of lower alkyl (meth)acrylates, from 2% to 50% by weight of fatty-chain allyl ether, and from 0 to 1% by weight of a crosslinking agent which is a well-known copolymerizable unsaturated polyethylenic monomer, for instance diallyl phthalate, allyl (meth)acrylate, divinylbenzene, (poly)ethylene glycol dimethacrylate or methylenebisacrylamide.

Among the latter polymers, those that are most particularly preferred are crosslinked terpolymers of methacrylic acid, of ethyl acrylate and of polyethylene glycol (10 EO) stearyl alcohol ether (Steareth-10), particularly those sold by the company Ciba under the names Salcare SC 80® and Salcare SC 90®, which are aqueous 30% emulsions of a crosslinked terpolymer of methacrylic acid, of ethyl acrylate and of steareth-10 allyl ether (40/50/10).
(b) those comprising i) at least one hydrophilic unit of unsaturated olefinic carboxylic acid type, and ii) at least one hydrophobic unit of the type such as a (C10-C30)alkyl ester of an unsaturated carboxylic acid.

(C10-C30)alkyl esters of unsaturated carboxylic acids that are of use for the invention comprise, for example, lauryl acrylate, stearyl acrylate, decyl acrylate, isodecyl acrylate and dodecyl acrylate, and the corresponding methacrylates: lauryl methacrylate, stearyl methacrylate, decyl methacrylate, isodecyl methacrylate and dodecyl methacrylate.

Anionic polymers of this type are described and prepared, for example, according to U.S. Pat. Nos. 3,915,921 and 4,509,949.

Among the anionic associative polymers of this type, use will be made more particularly of those constituted of 95% to 60% by weight of acrylic acid (hydrophilic unit), 4% to 40% by weight of C10-C30 alkyl acrylate (hydrophobic unit) and 0 to 6% by weight of crosslinking polymerizable monomer, or alternatively those constituted of 98% to 96% by weight of acrylic acid (hydrophilic unit), 1% to 4% by weight of C10-C30 alkyl acrylate (hydrophobic unit) and 0.1% to 0.6% by weight of crosslinking polymerizable monomer such as those described above.

Among said polymers above, those that are most particularly preferred according to the present invention are the products sold by the company Goodrich under the trade names Pemulen TR1®, Pemulen TR2®, Carbopol 13820, the product sold by the company Lubrizol under the trade name Carbopol ETD 2020 Polymer® (INCI name: Acrylates/C10-30 Alkyl Acrylate Crosspolymer), the product sold by the company S.E.P.C under the name Coatex SX®, and even more preferentially Carbopol ETD 2020 Polymer®.

Mention may also be made of the acrylic acid/lauryl methacrylate/vinylpyrrolidone terpolymer sold under the name Acrylidone LM by the company ISP.
(c) maleic anhydride/C30-C38 α-olefin/alkyl maleate terpolymers, such as the product (maleic anhydride/C30-C38 α-olefin/isopropyl maleate copolymer) sold under the name Performa V 1608® by the company Newphase Technologies.
(d) acrylic terpolymers comprising:
i) approximately 20% to 70% by weight of an α,β-monoethylenically unsaturated carboxylic acid [A],
ii) approximately 20% to 80% by weight of an α,β-monoethylenically unsaturated non-surfactant monomer other than [A],
iii) approximately 0.5% to 60% by weight of a nonionic monourethane which is the reaction product of a monohydric surfactant with a monoethylenically unsaturated monoisocyanate,
such as those described in patent application EP-A-0 173 109 and more particularly the terpolymer described in Example 3, namely a methacrylic acid/methyl acrylate/behenyl alcohol dimethyl-meta-isopropenylbenzylisocyanate ethoxylated (40 EO) terpolymer, as an aqueous 25% dispersion.
(e) copolymers comprising, among their monomers, an α,β-monoethylenically unsaturated carboxylic acid and an ester of an α,β-monoethylenically unsaturated carboxylic acid and of an oxyalkylenated fatty alcohol.

Preferentially, these compounds also comprise, as monomer, an ester of an α,β-monoethylenically unsaturated carboxylic acid and of a C1-C4 alcohol.

By way of example of this type of compound, mention may be made of Aculyn 22® sold by the company Rohm and Haas, which is an oxyalkylenated methacrylic acid/ethyl acrylate/stearyl methacrylate terpolymer, and also of Aculyn 88, also sold by the company Rohm and Haas, or even Aculyn 28® sold by the company Rohm and Haas, which is an oxyalkylenated methacrylic acid/ethyl acrylate/behenyl methacrylate terpolymer (INCI name: Acrylates/Beheneth-25 Methacrylate Copolymer), and also of the Novethix L-10 Polymer® sold by Lubrizol.
(f) amphiphilic polymers comprising at least one ethylenically unsaturated monomer comprising a sulfonic group, in free or partially or totally neutralized form and comprising at least one hydrophobic part. These polymers may be crosslinked or non-crosslinked. They are preferably crosslinked.

The ethylenically unsaturated monomers comprising a sulfonic group are particularly chosen from vinylsulfonic acid, styrenesulfonic acid, (meth)acrylamido(C1-C22)alkylsulfonic acids and N—(C1-C22)alkyl(meth)acrylamido(C1-C22)alkylsulfonic acids, for instance undecylacrylamidomethanesulfonic acid, and also partially or totally neutralized forms thereof.

Use will more preferentially be made of (meth)acrylamido(C1-C22)alkylsulfonic acids, for instance acrylamidomethanesulfonic acid, acrylamidoethanesulfonic acid, acrylamidopropanesulfonic acid, 2-acrylamido-2-methylpropanesulfonic acid, methacrylamido-2-methylpropanesulfonic acid, 2-acrylamido-n-butanesulfonic acid, 2-acrylamido-2,4,4-trimethylpentanesulfonic acid, 2-methacrylamidododecylsulfonic acid or 2-acrylamido-2,6-dimethyl-3-heptanesulfonic acid, and also partially or totally neutralized forms thereof.

Use will more particularly be made of 2-acrylamido-2-methylpropanesulfonic acid (AMPS), and also partially or totally neutralized forms thereof.

The polymers of this family may particularly be chosen from random amphiphilic AMPS polymers modified by reaction with a C6-C22 n-monoalkylamine or di-n-alkylamine, and such as those described in patent application WO 00/31154 (forming an integral part of the content of the description). These polymers may also contain other ethylenically unsaturated hydrophilic monomers chosen, for example, from (meth)acrylic acids, β-substituted alkyl derivatives thereof or esters thereof obtained with monoalcohols or mono- or polyalkylene glycols, (meth)acrylamides, vinylpyrrolidone, maleic anhydride, itaconic acid or maleic acid, or mixtures of these compounds.

The preferred polymers of this family are chosen from amphiphilic copolymers of AMPS and of at least one ethylenically unsaturated hydrophobic monomer.

These same copolymers may also contain one or more ethylenically unsaturated monomers that do not comprise a fatty chain, such as (meth)acrylic acids, β-substituted alkyl derivatives thereof or esters thereof obtained with monoalcohols or mono- or polyalkylene glycols, (meth)acrylamides, vinylpyrrolidone, maleic anhydride, itaconic acid or maleic acid, or mixtures of these compounds.

These copolymers are described particularly in patent application EP-A 750 899, U.S. Pat. No. 5,089,578 and in the following publications from Yotaro Morishima:
Self-assembling amphiphilic polyelectrolytes and their nanostructures, Chinese Journal of Polymer Science, Vol. 18, No. 40, (2000), 323-336;
Micelle formation of random copolymers of sodium 2-(acrylamido)-2-methylpropanesulfonate and a nonionic surfactant macromonomer in water as studied by fluorescence and dynamic light scattering, Macromolecules, Vol. 33, No. 10, (2000), 3694-3704;
Solution properties of micelle networks formed by non-ionic moieties covalently bound to an polyelectrolyte: salt effects on rheological behavior—Langmuir, Vol. 16, No. 12, (2000) 5324-5332;
Stimuli responsive amphiphilic copolymers of sodium 2-(acrylamido)-2-methylpropanesulfonate and associative macromonomers, Polym. Preprint, Div. Polym. Chem., 40(2), (1999), 220-221.

Among these polymers, mention may be made of:
crosslinked or non-crosslinked, neutralized or non-neutralized copolymers comprising from 15% to 60% by weight of AMPS units and from 40% to 85% by weight of (C8-C16)alkyl (meth)acrylamide units or of (C8-C16)alkyl (meth)acrylate units relative to the polymer, such as those described in application EP-A-750 899;
terpolymers comprising from 10 mol % to 90 mol % of acrylamide units, from 0.1 mol % to 10 mol % of AMPS units and from 5 mol % to 80 mol % of n-(C6-C18)alkylacrylamide units, such as those described in U.S. Pat. No. 5,089,578.

Mention may also be made of copolymers of totally neutralized AMPS and of dodecyl methacrylate, and also crosslinked and non-crosslinked copolymers of AMPS and of n-dodecylmethacrylamide, such as those described in the Morishima articles mentioned above.

Among the anionic associative polymers according to the invention, preference is given to polymers comprising i) at least one hydrophilic unit of unsaturated olefinic carboxylic acid type, and ii) at least one hydrophobic unit of the type such as a (C10-C30)alkyl ester of an unsaturated carboxylic acid (family b)), and copolymers comprising, among their monomers, an α,β-monoethylenically unsaturated carboxylic acid and an ester of an α,β-monoethylenically unsaturated carboxylic acid and of an oxyalkylenated fatty alcohol (family e)).

Among the cationic associative polymers, mention may be made of:
(a) cationic associative polyurethanes;
(b) the compound sold by the company Noveon under the name Aqua CC and which corresponds to the INCI name Polyacrylate-1 Crosspolymer.

Polyacrylate-1 Crosspolymer is the product of the polymerization of a mixture of monomers comprising:
a di(C1-C4 alkyl)amino (C1-C6 alkyl) methacrylate,
one or more C1-C30 alkyl esters of (meth)acrylic acid,
a polyethoxylated C10-C30 alkyl methacrylate (20-25 mol of ethylene oxide units),
a 30/5 polyethylene glycol/polypropylene glycol allyl ether,
a hydroxy (C2-C6 alkyl) methacrylate, and
an ethylene glycol dimethacrylate.
(c) quaternized (poly) hydroxyethylcelluloses modified with groups comprising at least one fatty chain, such as alkyl, arylalkyl or alkylaryl groups comprising at least 8 carbon atoms, or mixtures thereof. The alkyl radicals borne by the above quaternized celluloses or hydroxyethylcelluloses preferably comprise from 8 to 30 carbon atoms. The aryl radicals preferably denote phenyl, benzyl, naphthyl or anthryl groups. Examples of quaternized alkylhydroxyethylcelluloses containing C8-C30 fatty chains that may be indicated include the products Quatrisoft LM 200@, Quatrisoft LM-X 529-18-A®, Quatrisoft LM-X 529-18-B® (C12 alkyl) and Quatrisoft LM-X 529-8® (C18 alkyl) sold by the company Aqualon, and the products Crodacel QM®, Crodacel QL® (C12 alkyl) and Crodacel QS® (C18 alkyl) sold by the company Croda and the product Softcat SL 100@ sold by the company Aqualon.
(d) cationic polyvinyllactam polymers.

Such polymers are described, for example, in patent application WO-00/68282.

As cationic poly(vinyllactam) polymers according to the invention, use is particularly made of vinylpyrrolidone/dimethylaminopropylmethacrylamide/dodecyldimethylmethacrylamidopro pylammonium tosylate terpolymers, vinylpyrrolidone/dimethylaminopropylmethacrylamide/cocoyldimethylmethacrylamidopropy lammonium tosylate terpolymers, vinylpyrrolidone/dimethylaminopropylmethacrylamide/lauryldimethylmethacrylamidopropyl ammonium tosylate or chloride terpolymers.

The amphoteric associative polymers are preferably chosen from those comprising at least one noncyclic cationic unit. Even more particularly, preference is given to those prepared from or comprising 1 to 20 mol %, preferably 1.5 to 15 mol % and even more particularly 1.5 to 6 mol % of fatty-chain monomer relative to the total number of moles of monomers.

Amphoteric associative polymers according to the invention are described and prepared, for example, in patent application WO 98/44012.

Among the amphoteric associative polymers according to the invention, preference is given to acrylic acid/(meth)acrylamidopropyltrimethylammonium chloride/stearyl methacrylate terpolymers.

The associative polymers of nonionic type that may be used according to the invention are preferably chosen from:
(a) copolymers of vinylpyrrolidone and of fatty-chain hydrophobic monomers, of which mention may be made, by way of example:
the products Antaron V216® or Ganex V216® (vinylpyrrolidone/hexadecene copolymer), sold by the company ISP;
the products Antaron V220® or Ganex V220® (vinylpyrrolidone/eicosene copolymer), sold by the company ISP;
(b) copolymers of C1-C6 alkyl methacrylates or acrylates and of amphiphilic monomers comprising at least one fatty chain, for instance the oxyethylenated methyl acrylate/stearyl acrylate copolymer sold by the company Goldschmidt under the name Antil 208®. Or the copolymer with the INCI name “Acrylates/Beheneth-25 Methacrylate Copolymer”, such as the product Novethix L-10 polymer from Lubrizol.
(c) copolymers of hydrophilic methacrylates or acrylates and of hydrophobic monomers comprising at least one fatty chain, for instance the polyethylene glycol methacrylate/lauryl methacrylate copolymer.
(d) polyurethane polyethers comprising, in their chain, both hydrophilic blocks usually of polyoxyethylenated nature and hydrophobic blocks, which may be aliphatic sequences alone and/or cycloaliphatic and/or aromatic sequences.
(e) polymers with an aminoplast ether backbone containing at least one fatty chain, such as the Pure Thix® compounds sold by the company Sud-Chemie.
(f) celluloses or derivatives thereof, modified with groups comprising at least one fatty chain, such as alkyl, arylalkyl or alkylaryl groups or mixtures thereof in which the alkyl groups are of C8, and in particular:
nonionic alkylhydroxyethylcelluloses, such as the products Natrosol Plus Grade 330 CS and Polysurf 67 (C16 alkyl) sold by the company Aqualon;
nonionic nonoxynyl hydroxyethylcelluloses, such as the product Amercell HM-1500 sold by the company Amerchol;
nonionic alkylcelluloses, such as the product Bermocoll EHM 100 sold by the company Berol Nobel;
(g) associative guar derivatives, for instance hydroxypropyl guars modified with a fatty chain, such as the product Esaflor HM 22 (modified with a C22 alkyl chain) sold by the company Lamberti; the product Miracare XC 95-3 (modified with a C14 alkyl chain) and the product RE 205-146 (modified with a C20 alkyl chain) sold by Rhodia Chimie.

Preferably, the polyurethane polyethers comprise at least two hydrocarbon-based lipophilic chains having from 6 to 30 carbon atoms, separated by a hydrophilic block, it being possible for the hydrocarbon-based chains to be side chains or chains at the end of the hydrophilic block. In particular, it is possible for one or more pendent chains to be envisaged. In addition, the polymer may include a hydrocarbon-based chain at one end or at both ends of a hydrophilic block.

The polyurethane polyethers may be multiblock, in particular in triblock form. The hydrophobic blocks may be at each end of the chain (for example: triblock copolymer bearing a hydrophilic central block) or distributed both at the ends and in the chain (for example, multiblock copolymer). These same polymers may also be graft polymers or star polymers.

The fatty-chain nonionic polyurethane polyethers may be triblock copolymers, the hydrophilic block of which is a polyoxyethylenated chain including from 50 to 1000 oxyethylene groups. The nonionic polyurethane polyethers comprise a urethane bond between the hydrophilic blocks, giving rise to their name.

By extension, also included among the nonionic fatty-chain polyurethane polyethers are those in which the hydrophilic blocks are linked to the lipophilic blocks via other chemical bonds.

As examples of nonionic fatty-chain polyurethane polyethers that may be used in the invention, use may also be made of Rheolate 205® containing a urea function, sold by the company Rheox, or Rheolate® 208, 204 or 212, and also Acrysol RM 184®.

Mention may also be made of the product Elfacos T210® containing a C12-C14 alkyl chain, and the product Elfacos T212® containing a C18 alkyl chain, from Akzo.

Use may also be made of the product DW 1206B® from Rohm & Haas containing a C20 alkyl chain and containing a urethane bond, provided at a solids content of 20% in water.

Use may also be made of solutions or dispersions of these polymers, particularly in water or in an aqueous/alcoholic medium. Mention may be made, as examples of such polymers, of Rheolate® 255, Rheolate® 278 and Rheolate® 244, sold by the company Rheox. Use may also be made of the products DW 1206F and DW 1206J sold by the company Rohm & Haas.

The polyurethane polyethers that may be used according to the invention are in particular those described in the article by G. Fonnum, J. Bakke and Fk. Hansen—Colloid Polym. Sci., 271, 380-389 (1993).

It is even more particularly preferred to use a polyurethane polyether that may be obtained by polycondensation of at least three compounds comprising (i) at least one polyethylene glycol comprising from 150 to 180 mol of ethylene oxide, (ii) stearyl alcohol or decyl alcohol, and (iii) at least one diisocyanate.

Such polyether polyurethanes are sold in particular by the company Rohm & Haas under the names Aculyn 46® and Aculyn 44® [Aculyn 460 is a polycondensate of polyethylene glycol having 150 or 180 mol of ethylene oxide, of stearyl alcohol and of methylenebis(4-cyclohexyl isocyanate) (SMDI), at 15% by weight in a matrix of maltodextrin (4%) and water (81%); Aculyn 44® is a polycondensate of polyethylene glycol having 150 or 180 mol of ethylene oxide, of decyl alcohol and of methylenebis(4-cyclohexyl isocyanate) (SMDI), at 35% by weight in a mixture of propylene glycol (39%) and water (26%)].

Preferably, the associative polymer(s) are chosen from anionic associative polymers.

Preferably, the associative polymer(s) are chosen from acrylic acid or methacrylic acid homopolymers or copolymers.

More preferably still, the associative polymer(s) are chosen from polymers comprising i) at least one hydrophilic unit of unsaturated olefinic carboxylic acid type, and ii) at least one hydrophobic unit of the type such as a (C10-C30)alkyl ester of an unsaturated carboxylic acid, copolymers comprising, among their monomers, an α,β-monoethylenically unsaturated carboxylic acid and an ester of an α,β-monoethylenically unsaturated carboxylic acid and of an oxyalkylenated fatty alcohol, and mixtures thereof.

Preferably, the composition comprises one or more associative polymer(s) in a total content ranging from 0.01% to 15% by weight, more preferentially from 0.05% to 10% by weight, better still from 0.1% to 8% by weight, even better still from 0.2% to 5% by weight and indeed even from 0.3% to 3% by weight, relative to the total weight of the composition.

Preferably, the composition comprises one or more associative polymer(s) chosen from anionic associative polymers, preferably from acrylic acid or methacrylic acid homopolymers or copolymers, in a total content ranging from 0.01% to 15% by weight, more preferentially from 0.05% to 10% by weight, better still from 0.1% to 8% by weight, even better still from 0.2% to 5% by weight and indeed even from 0.3% to 3% by weight, relative to the total weight of the composition.

### Non-Associative Polysaccharides

The composition according to the invention also comprises one or more non-associative polysaccharides, which are thus other than the associative polymers above.

In the present invention, “polysaccharide” means a polymer constituted of sugar units. “Sugar unit” means an oxygen-bearing hydrocarbon-based compound containing several alcohol functions, with or without aldehyde or ketone functions, and which comprises at least 4 carbon atoms. The sugar units can be optionally modified by substitution, and/or by oxidation and/or by dehydration.

The sugar units that may be included in the composition of the polysaccharides of the invention are preferably derived from the following sugars: glucose, galactose, arabinose, rhamnose, mannose, xylose, fucose, anhydrogalactose, galacturonic acid, glucuronic acid, mannuronic acid, galactose sulfate, anhydrogalactose sulfate and fructose.

As non-associative polysaccharides, mention may particularly be made of the following polymers, alone or as a mixture:
a) tree or shrub exudates, including:
gum arabic (branched polymer of galactose, arabinose, rhamnose and glucuronic acid);
ghatti gum (polymer derived from arabinose, galactose, mannose, xylose and glucuronic acid);
karaya gum (polymer derived from galacturonic acid, galactose, rhamnose and glucuronic acid);
gum tragacanth (polymer of galacturonic acid, galactose, fucose, xylose and arabinose);
b) gums derived from algae, including:
agar (polymer derived from galactose and anhydrogalactose);
alginates (polymers of mannuronic acid and of glucuronic acid);
carrageenans and furcellerans (polymers of galactose sulfate and of anhydrogalactose sulfate);
c) gums derived from seeds or tubers, including:
guar gum (polymer of mannose and galactose);
locust bean gum (polymer of mannose and galactose);
fenugreek gum (polymer of mannose and galactose);
tamarind gum (polymer of galactose, xylose and glucose);
konjac gum (polymer of glucose and mannose);
d) microbial gums, including:
xanthan gum (polymer of glucose, mannose acetate, mannose/pyruvic acid and glucuronic acid);
gellan gum (polymer of partially acylated glucose, rhamnose and glucuronic acid);
scleroglucan gum (glucose polymer);
e) polymers extracted from plants, including:
celluloses (glucose polymers);
starches (glucose polymers) and
inulin.

These polymers may be physically or chemically modified. As physical treatment, mention may particularly be made of a heat treatment. As chemical treatment, mention may be made of esterification, etherification, amidation and oxidation reactions. These treatments make it possible to produce polymers that may particularly be nonionic, anionic or amphoteric.

It is in particular possible to modify/treat guar gums, locust bean gums, starches and celluloses.

The guar gums that may be used according to the invention may be modified with C1-C6 (poly) hydroxyalkyl groups. Among the C1-C6 (poly) hydroxyalkyl groups, mention may be made, by way of example, of hydroxymethyl, hydroxyethyl, hydroxypropyl and hydroxybutyl groups. These guar gums are well known in the prior art and may be prepared, for example, by reacting corresponding alkene oxides, for instance propylene oxides, with the guar gum so as to obtain a guar gum modified with hydroxypropyl groups. The degree of hydroxyalkylation preferably ranges from 0.4 to 1.2 and corresponds to the number of alkylene oxide molecules consumed by the number of free hydroxyl functions present on the guar gum.

Such guar gums optionally modified with hydroxyalkyl groups are sold, for example, under the trade names Jaguar HP8, Jaguar HP60 and Jaguar HP120 by the company Rhodia Chimie.

The botanical origin of the starches that may be used in the present invention may be cereals or tubers. Thus, the starches are chosen, for example, from corn starch, rice starch, oat starch, cassava starch, barley starch, potato starch, wheat starch, sorghum starch, pea starch or tapioca starch. It is also possible to use hydrolysates of the starches mentioned above. The starch is preferably derived from potato.

Use will preferentially be made of starch phosphates, particularly distarch phosphates or compounds rich in distarch phosphate, for instance the product sold under the references Prejel VA-70-T AGGL (gelatinized hydroxypropyl cassava distarch phosphate), or Prejel TK1 (gelatinized cassava distarch phosphate) or Prejel 200 (gelatinized acetyl cassava distarch phosphate) by the company Avebe, or Structure Zea from National Starch (gelatinized corn distarch phosphate).

According to the invention, amphoteric starches may also be used, these amphoteric starches comprising one or more anionic groups and one or more cationic groups. The anionic and cationic groups can be bonded to the same reactive site of the starch molecule or to different reactive sites; they are preferably bonded to the same reactive site. The anionic groups may be of carboxylic, phosphate or sulfate type, preferably carboxylic type. The cationic groups may be of primary, secondary, tertiary or quaternary amine type.

The polysaccharides that may be used according to the invention may be cellulose-based polymers.

According to the invention, “cellulose-based polymer” means any polysaccharide compound having, in its structure, sequences of glucose residues linked together via β-1,4 bonds; in addition to unsubstituted celluloses, the cellulose derivatives may be anionic, cationic, amphoteric or nonionic.

Cellulose-based polymers are also referred to as celluloses.

Thus, the cellulose-based polymers that may be used according to the invention may be chosen from unsubstituted celluloses, including those in a microcrystalline form, and cellulose ethers.

Among these cellulose-based polymers, a distinction is made between cellulose ethers, cellulose esters and cellulose ether/esters.

The cellulose esters include inorganic esters of cellulose (cellulose nitrates, sulfates, phosphates, etc.), organic esters of cellulose (cellulose monoacetates, triacetates, amidopropionates, acetatebutyrates, acetatepropionates or acetatetrimellitates, etc.), and mixed organic/inorganic esters of cellulose, such as cellulose acetatebutyrate sulfates and cellulose acetatepropionate sulfates. Among the cellulose ester ethers, mention may be made of hydroxypropylmethylcellulose phthalates and ethylcellulose sulfates.

Among the cellulose ethers, mention may be made of (C1-C4)alkylcelluloses, such as methylcelluloses and ethylcelluloses (for example Ethocel Standard 100 Premium from Dow Chemical); (poly) hydroxy (C1-C4)alkylcelluloses, such as hydroxymethylcelluloses, hydroxyethylcelluloses (for example Natrosol 250 HHR sold by Ashland) and hydroxypropylcelluloses (for example Klucel EF from Aqualon); mixed (poly) hydroxy (C1-C4)alkyl(C1-C4)alkylcelluloses, such as hydroxypropylmethylcelluloses (for example Methocel E4M from Chemical), hydroxyethylmethylcelluloses, Dow hydroxyethylethylcelluloses (for example Bermocoll E 481 FQ from Akzo Nobel) and hydroxybutylmethylcelluloses.

Among the anionic cellulose ethers, mention may be made of (poly) carboxy (C1-C4)alkylcelluloses and salts thereof. Mention may be made, by way of example, of carboxymethylcelluloses, carboxymethylmethylcelluloses (for example Blanose 7M from the company Aqualon) and carboxymethylhydroxyethylcelluloses, and the sodium salts thereof.

Among the cationic cellulose ethers, mention may be made of cationic cellulose derivatives such as cellulose copolymers or cellulose derivatives grafted with a water-soluble quaternary ammonium monomer, and described particularly in U.S. Pat. No. 4,131,576, such as (poly)hydroxy(C1-C4)alkylcelluloses, for instance hydroxymethyl-, hydroxyethyl- or hydroxypropylcelluloses, particularly grafted with a methacryloylethyltrimethylammonium, methacrylamidopropyltrimethylammonium or dimethyldiallylammonium salt. The commercial products corresponding to this definition are more particularly the products sold under the names Celquat® L 200 and Celquat® H 100 by the company National Starch.

Preferably, the non-associative polysaccharide(s) are chosen, alone or as a mixture, from celluloses, guar gum, starches, preferentially from celluloses.

Better still, the non-associative polysaccharides are chosen, alone or as a mixture, from cellulose ethers, cellulose esters and cellulose ester ethers, and preferably from cellulose ethers.

Particularly preferably, the non-associative polysaccharide(s) are chosen from (C1-C4)alkylcelluloses such as methylcelluloses and ethylcelluloses, (poly)hydroxy(C1-C4)alkylcelluloses such as hydroxymethylcelluloses, hydroxyethylcelluloses and hydroxypropylcelluloses; and mixed (poly)hydroxy(C1-C4)alkyl(C1-C4)alkylcelluloses such as hydroxypropylmethylcelluloses, hydroxyethylmethylcelluloses, hydroxyethylethylcelluloses and hydroxybutylmethylcelluloses.

Preferably, the composition according to the invention comprises the non-associative polysaccharide(s) in a total amount ranging from 0.01% to 15% by weight, more preferentially from 0.05% to 10% by weight, better still from 0.1% to 8% by weight, even better still from 0.2% to 5% by weight and indeed even from 0.3% to 3% by weight, relative to the total weight of the composition according to the invention.

Preferentially, the composition according to the invention comprises the non-associative polysaccharide(s) chosen from cellulose-based polymers in a total amount ranging from 0.01% to 15% by weight, more preferentially from 0.05% to 10% by weight, better still from 0.1% to 8% by weight, even better still from 0.2% to 5% by weight and indeed even from 0.3% to 3% by weight, relative to the total weight of the composition according to the invention.

More preferentially, the composition according to the invention comprises the non-associative polysaccharide(s) chosen from cellulose ethers in a total amount ranging from 0.01% to 15% by weight, more preferentially from 0.05% to 10% by weight, better still from 0.1% to 8% by weight, even better still from 0.2% to 5% by weight and indeed even from 0.3% to 3% by weight, relative to the total weight of the composition according to the invention.

Hydrocarbon(s) with a Melting Point of Greater than 25° C.

The composition according to the invention also comprises one or more hydrocarbons with a melting point of greater than 25° C.

“Hydrocarbon with a melting point of greater than 25° C.” means a hydrocarbon having a melting point of greater than 25° C. at atmospheric pressure (1.013×10⁵ Pa).

For the purposes of the present invention, the melting point corresponds to the temperature of the most endothermic peak observed on thermal analysis (differential scanning calorimetry or DSC) as described in the standard ISO 11357-3; 1999. The melting point may be measured using a differential scanning calorimeter (DSC), for example the calorimeter sold under the name “MDSC 2920” by the company TA Instruments. In the present application, all melting points are determined at atmospheric pressure (1.013×10⁵ Pa).

The hydrocarbons according to the invention are constituted of carbon atoms and hydrogen atoms, i.e. they only contain carbon atoms and hydrogen atoms. They preferably comprise at least 30 carbon atoms, preferentially at least 35 carbon atoms, and better still at least 40 carbon atoms.

The hydrocarbons with a melting point of greater than 25° C. may be linear or branched, preferably linear.

Preferably, the hydrocarbons with a melting point of greater than 25° C. are saturated.

Preferably, the hydrocarbons according to the invention have a melting point of greater than or equal to 30° C., preferably greater than or equal to 50° C., preferentially greater than or equal to 70° C., better still greater than or equal to 80° C. More preferentially, the hydrocarbons according to the invention have a melting point ranging from 85 to 150° C., better still from 90 to 120° C.

The number-average molar mass (Mn) of the hydrocarbons according to the invention is preferably between approximately 400 and 2000, more particularly between approximately 400 and 1000, and more preferentially between 500 and 700.

The number-average molecular masses of these hydrocarbons may be measured by gel permeation chromatography (GPC) at ambient temperature (25° C.), as polystyrene equivalent. The columns used are μ styragel columns. The eluent is THF and the flow rate is 1 ml/min. 200 μl of a 0.5% by weight solution of silicone in THF are injected. Detection is performed by refractometry and UV-metry.

Preferably, the hydrocarbons according to the invention are chosen from waxes.

For the purposes of the present invention, a wax is a lipophilic compound, which is solid at 25° C. and atmospheric pressure, with a reversible solid/liquid change of state, having a melting point of greater than approximately 40° C. and which may be up to 200° C., and having anisotropic crystal organization in the solid state. In general, the size of the wax crystals is such that the crystals diffract and/or scatter light, giving the composition that comprises them a more or less opaque cloudy appearance. By bringing the wax to its melting point, it is possible to make it miscible with oils and to form a microscopically homogeneous mixture, but on returning the temperature of the mixture to room temperature, recrystallization of the wax, which is microscopically and macroscopically detectable (opalescence), is obtained.

Preferably, the hydrocarbons according to the invention are chosen from microcrystalline waxes, polyethylene waxes, Fischer-Tropsch waxes, paraffin waxes, ozokerite, and mixtures thereof.

According to a preferred embodiment, the hydrocarbon(s) with a melting point of greater than 25° C. are chosen from ethylene homopolymers, also referred to as polyethylenes.

Preferably, the hydrocarbon(s) according to the invention are chosen from ethylene homopolymers with a melting point of greater than or equal to 30° C., preferably greater than or equal to 50° C., preferentially greater than or equal to 70° C., better still greater than or equal to 80° C., preferentially ranging from 85 to 150° C., better still from 90 to 120° C.

More preferably still, the hydrocarbons according to the invention are chosen from polyethylene waxes.

According to a particularly preferred embodiment, the hydrocarbons according to the invention are chosen from polyethylene waxes with a melting point of greater than or equal to 80° C.

Among the polyethylene waxes which can be used according to the invention, mention can particularly be made of that sold under the name CIRE POLYETHYLENE A C 1702 by the company HONEYWELL, and those sold under the names PERFORMALENE® 500-L POLYETHYLENE, PERFORMALENE® 400 POLYETHYLENE, PERFORMALENE® 655 POLYETHYLENE, PERFORMALENE® SCRUB BEADS, PERFORMALENE® SE/2 POLYETHYLENE, POLYWAX® 725 POLYETHYLENE, POLYWAX® 850 POLYETHYLENE, POLYWAX® 1000 POLYETHYLENE by the company NUCERA SOLUTIONS.

Preferably, the total content of hydrocarbons with a melting point of greater than or equal to 25° C. ranges from 0.1% to 30% by weight, preferably from 0.5% to 20% by weight, more preferentially from 1% to 10% by weight, better still from 1.5% to 5% by weight, relative to the total weight of the composition.

Preferably, the total content of hydrocarbons with a melting point of greater than 25° C. chosen from ethylene homopolymers ranges from 0.1% to 30% by weight, preferably from 0.5% to 20% by weight, more preferentially from 1% to 10% by weight, better still from 1.5% to 5% by weight, relative to the total weight of the composition.

Preferably, the total content of hydrocarbon(s) with a melting point of greater than 25° C. chosen from polyethylene waxes ranges from 0.1% to 30% by weight, preferably from 0.5% to 20% by weight, more preferentially from 1% to 10% by weight, better still from 1.5% to 5% by weight, relative to the total weight of the composition.

Fatty Substances Other than the Hydrocarbon(s) with a Melting Point of Greater than 25° C.

The composition according to the invention may also comprise one or more fatty substances other than the hydrocarbons according to the invention described previously.

Preferably, the composition according to the invention comprises one or more fatty substances other than the hydrocarbons with a melting point of greater than 25° C.

“Fatty substance” means an organic compound that is insoluble in water at 25° C. and at atmospheric pressure (1.013×10⁵ Pa) (solubility of less than 5% by weight, preferably less than 1% by weight, even more preferentially less than 0.1% by weight). They have in their structure at least one hydrocarbon-based chain comprising at least 6 carbon atoms and/or a sequence of at least two siloxane groups. In addition, the fatty substances are generally soluble in organic solvents under the same temperature and pressure conditions, for instance chloroform, dichloromethane, carbon tetrachloride, ethanol, benzene, toluene, tetrahydrofuran (THF), liquid petroleum jelly or decamethylcyclopentasiloxane.

Advantageously, the fatty substances that may be used in the present invention are neither (poly)oxyalkylenated nor (poly)glycerolated.

Preferably, useful fatty substances according to the invention are nonsilicone.

“Nonsilicone fatty substance” means a fatty substance not containing any Si—O bonds, and “silicone fatty substance” means a fatty substance containing at least one Si—O bond.

Useful fatty substances according to the invention may be liquid fatty substances (or oils) and/or solid fatty substances. “Liquid fatty substance” means a fatty substance having a melting point of less than or equal to 25° C. at atmospheric pressure (1.013×10⁵ Pa). “Solid fatty substance” means a fatty substance having a melting point of greater than 25° C. at atmospheric pressure (1.013×10⁵ Pa).

For the purposes of the present invention, the melting point corresponds to the temperature of the most endothermic peak observed on thermal analysis (differential scanning calorimetry or DSC) as described in the standard ISO 11357-3; 1999. The melting point may be measured using a differential scanning calorimeter (DSC), for example the calorimeter sold under the name “MDSC 2920” by the company TA Instruments. In the present application, all melting points are determined at atmospheric pressure (1.013×10⁵ Pa).

More particularly, the liquid fatty substance(s) according to the invention are chosen from C6 to C16 liquid hydrocarbons, liquid hydrocarbons comprising more than 16 carbon atoms, nonsilicone oils of animal origin, oils of triglyceride type of plant or synthetic origin, fluoro oils, liquid fatty alcohols, liquid fatty acid and/or fatty alcohol esters other than triglycerides, silicone oils, and mixtures thereof.

It is recalled that the fatty alcohols, esters and acids more particularly have at least one saturated or unsaturated, linear or branched hydrocarbon-based group comprising from 6 to 40 and better still from 8 to 30 carbon atoms, which is optionally substituted, in particular with one or more hydroxyl groups (in particular 1 to 4). If they are unsaturated, these compounds may comprise one to three conjugated or unconjugated carbon-carbon double bonds.

As regards the C6 to C16 liquid hydrocarbons, they may be linear, branched, or optionally cyclic, and are preferably chosen from alkanes. Examples that may be mentioned include hexane, cyclohexane, undecane, dodecane, isododecane, tridecane or isoparaffins, such as isohexadecane or isodecane, and mixtures thereof.

The liquid hydrocarbons comprising more than 16 carbon atoms may be linear or branched, and of mineral or synthetic origin, and are preferably chosen from liquid paraffins or liquid petroleum jelly (INCI name: mineral oil or paraffinum liquidum), polydecenes, hydrogenated polyisobutene such as Parleam®, and mixtures thereof.

A hydrocarbon-based oil of animal origin that may be mentioned is perhydrosqualene.

The triglyceride oils of plant or synthetic origin are preferably chosen from liquid fatty acid triglycerides comprising from 6 to 30 carbon atoms, for instance heptanoic or octanoic acid triglycerides, or alternatively, for example, sunflower oil, corn oil, soybean oil, marrow oil, grapeseed oil, sesame oil, hazelnut oil, apricot oil, macadamia nut oil, arara oil, castor oil, avocado oil, caprylic/capric acid triglycerides, for instance those sold by the company Stearineries Dubois or those sold under the names Miglyol® 810, 812 and 818 by the company Dynamit Nobel, jojoba oil and shea butter oil, and mixtures thereof.

As regards the fluoro oils, they may be chosen from perfluoromethylcyclopentane and perfluoro-1,3-dimethylcyclohexane, sold under the names Flutec® PC1 and Flutec® PC3 by the company BNFL Fluorochemicals; perfluoro-1,2-dimethylcyclobutane; perfluoroalkanes such as dodecafluoropentane and tetradecafluorohexane, sold under the names PF 5050® and PF 5060® by the company 3M, or bromoperfluorooctyl sold under the name Foralkyl® by the company Atochem; nonafluoromethoxybutane and nonafluoroethoxyisobutane; perfluoromorpholine derivatives such as 4-trifluoromethylperfluoromorpholine sold under the name PF 5052® by the company 3M.

The liquid fatty alcohols that are suitable for use in the invention are more particularly chosen from linear or branched, saturated or unsaturated alcohols, preferably unsaturated or branched alcohols, comprising from 6 to 40 carbon atoms, preferably from 8 to 30 carbon atoms. Examples that may be mentioned include octyldodecanol, 2-butyloctanol, 2-hexyldecanol, 2-undecylpentadecanol, isostearyl alcohol, oleyl alcohol, linolenyl alcohol, ricinoleyl alcohol, undecylenyl alcohol and linoleyl alcohol, and mixtures thereof.

As regards the liquid esters of fatty acids and/or of fatty alcohols, other than the triglycerides mentioned previously, mention may particularly be made of esters of saturated or unsaturated, linear C1 to C26 or branched C3 to C26 aliphatic mono- or polyacids and of saturated or unsaturated, linear C1 to C26 or branched C3 to C26 aliphatic mono- or polyalcohols, the total carbon number of the esters being greater than or equal to 6 and more advantageously greater than or equal to 10.

Preferably, for the esters of monoalcohols, at least one from among the alcohol and the acid from which the esters of the invention are derived is branched.

Among the monoesters, mention may be made of dihydroabietyl behenate; octyldodecyl behenate; isocetyl behenate; isostearyl lactate; lauryl lactate; linoleyl lactate; oleyl lactate; isostearyl octanoate; isocetyl octanoate; octyl octanoate; decyl oleate; isocetyl isostearate; isocetyl laurate; isocetyl stearate; isodecyl octanoate; isodecyl oleate; isononyl isononanoate; isostearyl palmitate; methyl acetyl ricinoleate; octyl isononanoate; 2-ethylhexyl isononanoate; octyldodecyl erucate; oleyl erucate; ethyl and isopropyl palmitates, such as 2-ethylhexyl palmitate, 2-octyldecyl palmitate; alkyl myristates such as isopropyl myristate; isobutyl stearate; 2-hexyldecyl laurate, and mixtures thereof.

Preferably, among the monoesters of monoacids and of monoalcohols, use will be made of ethyl and isopropyl palmitates, alkyl myristates such as isopropyl myristate or ethyl myristate, isocetyl stearate, 2-ethylhexyl isononanoate, isodecyl neopentanoate and isostearyl neopentanoate, and mixtures thereof.

Still within the context of this variant, esters of C4-C22 dicarboxylic or tricarboxylic acids and of C1-C22 alcohols and esters of monocarboxylic, dicarboxylic or tricarboxylic acids and of C2-C26 dihydroxy, trihydroxy, tetrahydroxy or pentahydroxy alcohols may also be used.

Mention may particularly be made of: diethyl sebacate; diisopropyl sebacate; diisopropyl adipate; di-n-propyl adipate; dioctyl adipate; diisostearyl adipate; dioctyl maleate; glyceryl undecylenate; octyldodecyl stearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate; propylene glycol dicaprate; tridecyl erucate; triisopropyl citrate; triisostearyl citrate; glyceryl trilactate; glyceryl trioctanoate; trioctyldodecyl citrate; trioleyl citrate; propylene glycol dioctanoate; neopentyl glycol diheptanoate; diethylene glycol diisononanoate; and polyethylene glycol distearates, and mixtures thereof.

The composition may also comprise, as fatty ester, sugar esters and diesters of C6 to C30 and preferably C12 to C22 fatty acids. It is recalled that “sugar” means oxygen-containing hydrocarbon-based compounds bearing several alcohol functions, with or without aldehyde or ketone functions, and which comprise at least 4 carbon atoms. These sugars may be monosaccharides, oligosaccharides or polysaccharides.

Examples of suitable sugars that may be mentioned include sucrose, glucose, galactose, ribose, fucose, maltose, fructose, mannose, arabinose, xylose and lactose, and derivatives thereof, particularly alkyl derivatives, such as methyl derivatives, for instance methylglucose.

The sugar esters of fatty acids may particularly be chosen from the group comprising the esters or mixtures of esters of sugars described above and of linear or branched, saturated or unsaturated C6 to C30 and preferably C12 to C22 fatty acids. If they are unsaturated, these compounds may comprise one to three conjugated or unconjugated carbon-carbon double bonds.

The esters according to this variant may also be chosen from mono-, di-, tri- and tetraesters, polyesters, and mixtures thereof.

These esters may be, for example, oleates, laurates, palmitates, myristates, behenates, cocoates, stearates, linoleates, linolenates, caprates, arachidonates or mixtures thereof, particularly such as the mixed oleo-palmitate, oleo-stearate and palmito-stearate esters.

More particularly, use is made of monoesters and diesters and notably sucrose, glucose or methylglucose mono- or di-oleates, stearates, behenates, oleopalmitates, linoleates, linolenates and oleostearates, and mixtures thereof.

An example that may be mentioned is the product sold under the name Glucate® DO by the company Amerchol, which is a methylglucose dioleate.

Preferably, use will be made of a liquid ester of a monoacid and of a monoalcohol.

The silicone oils that may be used in the composition according to the present invention may be volatile or nonvolatile, cyclic, linear or branched silicone oils, which are unmodified or modified with organic groups, and preferably have a viscosity from 5×10⁻⁶ to 2.5 m²/s at 25° C., and preferably 1×10⁻⁵ to 1 m²/s.

Preferably, the silicone oils are chosen from polydialkylsiloxanes, particularly polydimethylsiloxanes (PDMS), and liquid polyorganosiloxanes comprising at least one aryl group.

These silicone oils may also be organomodified. The organomodified silicone oils that may be used in accordance with the invention are preferably liquid silicones as defined previously that comprise in their structure one or more organofunctional groups attached via a hydrocarbon-based group, chosen, for example, from amine groups and alkoxy groups.

Organopolysiloxanes are defined in greater detail in Walter Noll's Chemistry and Technology of Silicones (1968), Academic Press. They may be volatile or nonvolatile.

When they are volatile, the silicone oils are more particularly chosen from those with a boiling point of between 60° C. and 260° C., and even more particularly from:
(i) cyclic polydialkylsiloxanes comprising from 3 to 7 and preferably from 4 to 5 silicon atoms. These are, for example, octamethylcyclotetrasiloxane, particularly sold under the name Volatile Silicone® 7207 by Union Carbide or Silbione® 70045 V2 by Rhodia, decamethylcyclopentasiloxane sold under the name Volatile Silicone® 7158 by Union Carbide, and Silbione® 70045 V5 by Rhodia, and mixtures thereof.

Mention may also be made of cyclocopolymers of the dimethylsiloxane/methylalkylsiloxane type, such as Volatile Silicone® FZ 3109 sold by the company Union Carbide.

Mention may also be made of mixtures of cyclic polydialkylsiloxanes with organic silicon-derived compounds, such as the mixture of octamethylcyclotetrasiloxane and tetra(trimethylsilyl)pentaerythritol (50/50) and the mixture of octamethylcyclotetrasiloxane and oxy-1,1′-bis(2,2,2′,2′,3,3′-hexatrimethylsilyloxy)neopentane;

(ii) linear volatile polydialkylsiloxanes having 2 to 9 silicon atoms and having a viscosity of less than or equal to 5×10⁻⁶ m²/s at 25° C. An example is decamethyltetrasiloxane, particularly sold under the name SH 200 by the company Toray Silicone. Silicones falling within this category are also described in the article published in Cosmetics and Toiletries, Vol. 91, January 76, pages 27-32, Todd & Byers Volatile Silicone Fluids for Cosmetics.

Nonvolatile polydialkylsiloxanes are preferably used.

These silicone oils are more particularly chosen from polydialkylsiloxanes, among which mention may be made mainly of polydimethylsiloxanes bearing trimethylsilyl end groups. The viscosity of the silicones is measured at 25° C. according to the standard ASTM 445 Appendix C.

Mention may be made, among these polydialkylsiloxanes, in a non-limiting way, of the following commercial products:
the Silbione® oils of the 47 and 70 047 series or the Mirasil® oils sold by Rhodia, for instance the oil 70 047 V 500 000;
the oils of the Mirasil® series sold by Rhodia;
the oils of the 200 series from the company Dow Corning, such as DC200, with a viscosity of 60 000 mm²/s;
the Viscasil® oils from General Electric and certain oils of the SF series (SF 96, SF 18) from General Electric.

Mention may also be made of polydimethylsiloxanes bearing dimethylsilanol end groups, known under the name dimethiconol (CTFA), such as the oils of the 48 series from the company Rhodia.

The organomodified silicones that may be used in accordance with the invention are silicones as defined previously that comprise in their structure one or more organofunctional groups attached via a hydrocarbon-based group.

As regards the liquid polyorganosiloxanes comprising at least one aryl group, they may particularly be polydiphenylsiloxanes, and polyalkylarylsiloxanes functionalized by the abovementioned organofunctional groups.

The polyalkylarylsiloxanes are chosen particularly from linear and/or branched polydimethyl/methylphenylsiloxanes or polydimethyl/diphenylsiloxanes with a viscosity ranging from 1×10⁻⁵ to 5×10⁻² m²/s at 25° C.

Among these polyalkylarylsiloxanes, examples that may be mentioned include the products sold under the following names:
the Silbione® oils of the 70 641 series from Rhodia;
the oils of the Rhodorsil® 70 633 and 763 series from Rhodia;
the oil Dow Corning 556 Cosmetic Grade Fluid from Dow Corning;
the silicones of the PK series from Bayer, such as the product PK20;
the silicones of the PN and PH series from Bayer, such as the products PN1000 and PH1000;
certain oils of the SF series from General Electric, such as SF 1023, SF 1154, SF 1250 and SF 1265.

Among the organomodified silicones, mention may be made of polyorganosiloxanes comprising:
substituted or unsubstituted amine groups, such as the products sold under the names GP 4 Silicone Fluid and GP 7100 by the company Genesee or the products sold under the names Q2 8220 and Dow Corning 929 or 939 by the company Dow Corning. The substituted amine groups are in particular C1 to C4 aminoalkyl groups;
alkoxylated groups,
hydroxyl groups.

The solid fatty substances according to the invention preferably have a viscosity of greater than 2 Pa·s, measured at 25° C. and at a shear rate of 1 s⁻¹.

The solid fatty substance(s) are preferably chosen from solid fatty acids, solid fatty alcohols, solid esters of fatty acids and/or of fatty alcohols, waxes, ceramides and mixtures thereof.

“Fatty acid” means a long-chain carboxylic acid comprising from 6 to 40 carbon atoms, preferably from 8 to 30 carbon atoms. The solid fatty acids according to the invention preferentially comprise from 10 to 30 carbon atoms and better still from 14 to 22 carbon atoms. They can optionally be hydroxylated. These fatty acids are neither oxyalkylenated nor glycerolated.

The solid fatty acids that may be used in the present invention are particularly chosen from myristic acid, cetylic acid, stearylic acid, palmitic acid, arachidic acid, stearic acid, lauric acid, behenic acid, 12-hydroxystearic acid, and mixtures thereof.

Particularly preferably, the solid fatty acid(s) are chosen from stearic acid, myristic acid and palmitic acid.

“Fatty alcohol” means a long-chain aliphatic alcohol comprising from 6 to 40 carbon atoms, preferably from 8 to 30 carbon atoms, and comprising at least one hydroxyl group OH. These fatty alcohols are neither oxyalkylenated nor glycerolated.

The solid fatty alcohols can be saturated or unsaturated and linear or branched, and they comprise from 8 to 40 carbon atoms, preferably from 10 to 30 carbon atoms. Preferably, the solid fatty alcohols are of structure R—OH, with R denoting a linear alkyl group, optionally substituted with one or more hydroxyl groups, comprising from 8 to 40, preferentially from 10 to 30, carbon atoms, better still from 10 to 30, indeed even from 12 to 24, carbon atoms, even better still from 14 to 22 carbon atoms.

The solid fatty alcohols that may be used are preferably chosen from saturated or unsaturated, linear or branched, preferably linear and saturated, (mono) alcohols comprising from 8 to 40 carbon atoms, better still from 10 to 30, indeed even from 12 to 24, carbon atoms, even better still from 14 to 22 carbon atoms.

The solid fatty alcohols that may be used may be chosen from, alone or as a mixture: myristyl alcohol (or 1-tetradecanol); cetyl alcohol (or 1-hexadecanol); stearyl alcohol (or 1-octadecanol); arachidyl alcohol (or 1-eicosanol); behenyl alcohol (or 1-docosanol); lignoceryl alcohol (or 1-tetracosanol); ceryl alcohol (or 1-hexacosanol); montanyl alcohol (or 1-octacosanol); and myricyl alcohol (or 1-triacontanol).

Preferentially, the solid fatty alcohol is chosen from cetyl alcohol, stearyl alcohol, behenyl alcohol, myristyl alcohol, arachidyl alcohol, and mixtures thereof, such as cetylstearyl alcohol or cetearyl alcohol. Particularly preferably, the solid fatty alcohol is chosen from cetylstearyl or cetearyl alcohol and cetyl alcohol.

The solid esters of a fatty acid and/or of a fatty alcohol that may be used are preferably chosen from esters derived from a C9-C26 carboxylic fatty acid and/or from a C9-C26 fatty alcohol.

Preferably, these solid fatty esters are esters of a linear or branched, saturated carboxylic acid comprising at least 10 carbon atoms, preferably from 10 to 30 carbon atoms and more particularly from 12 to 24 carbon atoms, and of a linear or branched, saturated monoalcohol comprising at least 10 carbon atoms, preferably from 10 to 30 carbon atoms and more particularly from 12 to 24 carbon atoms. The saturated carboxylic acids may optionally be hydroxylated, and are preferably monocarboxylic acids.

Esters of C4-C22 dicarboxylic or tricarboxylic acids and of C1-C22 alcohols and esters of monocarboxylic, dicarboxylic or tricarboxylic acids and of C2-C26 dihydroxy, trihydroxy, tetrahydroxy or pentahydroxy alcohols may also be used.

Mention may particularly be made of octyldodecyl behenate, isocetyl behenate, cetyl lactate, stearyl octanoate, octyl octanoate, cetyl octanoate, decyl oleate, hexyl stearate, octyl stearate, myristyl stearate, cetyl stearate, stearyl stearate, octyl pelargonate, cetyl myristate, myristyl myristate, stearyl myristate, diethyl sebacate, diisopropyl sebacate, diisopropyl adipate, di-n-propyl adipate, dioctyl adipate, dioctyl maleate, octyl palmitate, myristyl palmitate, cetyl palmitate, stearyl palmitate, and mixtures thereof.

Preferably, the solid esters of a fatty acid and/or of a fatty alcohol are chosen from C9-C26 alkyl palmitates, particularly myristyl, cetyl or stearyl palmitates; C9-C26 alkyl myristates, such as cetyl myristate, stearyl myristate and myristyl myristate; and C9-C26 alkyl stearates, particularly myristyl, cetyl and stearyl stearates; and mixtures thereof.

For the purposes of the present invention, a wax is a lipophilic compound, which is solid at 25° C. and atmospheric pressure, with a reversible solid/liquid change of state, having a melting point of greater than approximately 40° C. and which may be up to 200° C., and having anisotropic crystal organization in the solid state. In general, the size of the wax crystals is such that the crystals diffract and/or scatter light, giving the composition that comprises them a more or less opaque cloudy appearance. By bringing the wax to its melting point, it is possible to make it miscible with oils and to form a microscopically homogeneous mixture, but on returning the temperature of the mixture to room temperature, recrystallization of the wax, which is microscopically and macroscopically detectable (opalescence), is obtained.

In particular, the waxes that are suitable for use in the invention may be chosen from waxes of animal or plant origin, nonsilicone synthetic waxes, and mixtures thereof.

Mention may particularly be made of beeswax, particularly of organic origin, lanolin wax and Chinese insect waxes; rice bran wax, carnauba wax, candelilla wax, ouricury wax, esparto grass wax, berry wax, shellac wax, Japan wax and sumac wax; montan wax, orange wax, lemon wax, and waxy copolymers, and also esters thereof.

Mention may also be made of the waxes obtained by catalytic hydrogenation of animal or plant oils having linear or branched C8-C32 fatty chains. Among these waxes, mention may particularly be made of isomerized jojoba oil such as trans-isomerized partially hydrogenated jojoba oil, particularly the product manufactured or sold by the company Desert Whale under the commercial reference Iso-Jojoba-500, hydrogenated sunflower oil, hydrogenated castor oil, hydrogenated coconut kernel oil, hydrogenated lanolin oil and bis(1,1,1-trimethylolpropane) tetrastearate, particularly the product sold under the name Hest 2T-4S® by the company Heterene.

The waxes obtained by hydrogenation of castor oil esterified with cetyl alcohol, such as those sold under the names Phytowax Castor 16L64® and 22L73® by the company Sophim, may also be used.

A wax that may also be used is a C20 to C40 alkyl (hydroxystearyloxy) stearate (the alkyl group comprising from 20 to 40 carbon atoms), alone or as a mixture. Such a wax is particularly sold under the names Kester Wax K 82 PR, Hydroxypolyester K 82 P® and Kester Wax K 80 P® by the company Koster Keunen.

It is also possible to use microwaxes in the compositions; mention may particularly be made of microwaxes of carnauba, such as that sold under the name MicroCare 3500 by the company Micro Powders, and polytetrafluoroethylene microwaxes, such as those sold under the names Microslip 519® and 519 L® by the company Micro Powders.

The waxes are preferably chosen from plant waxes, such as cocoa butter or cork fiber or sugar cane waxes, olive tree wax, rice wax, hydrogenated jojoba wax, ouricury wax, carnauba wax, candelilla wax, esparto grass wax, or absolute waxes of flowers, such as the essential wax of blackcurrant blossom sold by Bertin (France); waxes of animal origin, such as beeswaxes or modified beeswaxes (cera bellina), spermaceti, lanolin wax and lanolin derivatives; and mixtures thereof.

Ceramides, or ceramide analogues, such as glycoceramides, that may be used in the compositions according to the invention, are known; mention may in particular be made of ceramides of classes I, II, III and V according to the Dawning classification.

The ceramides or analogues thereof that may be used preferably correspond to the following formula: R3CH(OH)CH(CH2OR2)(NHCOR1), in which:
R1 denotes a linear or branched, saturated or unsaturated alkyl group, derived from C14-C30 fatty acids, it being possible for this group to be substituted with a hydroxyl group in the alpha position, or a hydroxyl group in the omega position esterified with a saturated or unsaturated C16-C30 fatty acid;
R2 denotes a hydrogen atom or a (glycosyl)n group, a (galactosyl)m group or a sulfogalactosyl group, in which n is an integer ranging from 1 to 4 and m is an integer ranging from 1 to 8;
R3 denotes a C15-C26 hydrocarbon-based group saturated or unsaturated in the alpha position, it being possible for this group to be substituted with one or more C1-C14 alkyl groups; it being understood that in the case of natural ceramides or glycoceramides, R3 may also denote a C15-C26 α-hydroxyalkyl group, the hydroxyl group being optionally esterified with a C16-C30 α-hydroxy acid.

The ceramides that are more particularly preferred are the compounds for which R1 denotes a saturated or unsaturated alkyl derived from C16-C22 fatty acids; R2 denotes a hydrogen atom; and R3 denotes a saturated linear C15 group.

Preferentially, use is made of ceramides for which R1 denotes a saturated or unsaturated alkyl group derived from C14-C30 fatty acids; R2 denotes a galactosyl or sulfogalactosyl group; and R3 denotes a —CH═CH—(CH2)12-CH3 group.

Use may also be made of the compounds for which R1 denotes a saturated or unsaturated alkyl radical derived from C12-C22 fatty acids; R2 denotes a galactosyl or sulfogalactosyl radical and R3 denotes a saturated or unsaturated C12-C22 hydrocarbon-based radical and preferably a —CH═CH—(CH2)12-CH3 group.

As compounds that are particularly preferred, mention may also be made of 2-N-linoleoylaminooctadecane-1,3-diol; 2-N-oleoylaminooctadecane-1,3-diol; 2-N-palmitoylaminooctadecane-1,3-diol; 2-N-stearoylaminooctadecane-1,3-diol; 2-N-behenoylaminooctadecane-1,3-diol; 2-N-[2-hydroxypalmitoyl]aminooctadecane-1,3-diol; 2-N-stearoylaminooctadecane-1,3,4-triol and in particular N-stearoylphytosphingosine, 2-N-palmitoylaminohexadecane-1,3-diol, N-linoleoyldihydrosphingosine, N-oleoyldihydrosphingosine, N-palmitoyldihydrosphingosine, N-stearoyldihydrosphingosine, and N-behenoyldihydrosphingosine, N-docosanoyl-N-methyl-D-glucamine, cetylic acid N-(2-hydroxyethyl)-N-(3-cetyloxy-2-hydroxypropyl)amide and bis(N-hydroxyethyl-N-cetyl) malonamide; and mixtures thereof. Use will preferably be made of N-oleoyldihydrosphingosine.

The solid fatty substances are preferably chosen from solid fatty acids, solid fatty alcohols, waxes and mixtures thereof.

According to a preferred embodiment, the composition according to the invention comprises at least one liquid fatty substance, preferentially chosen from liquid hydrocarbons containing more than 16 carbon atoms, plant oils, liquid fatty alcohols, liquid fatty esters, silicone oils and mixtures thereof.

According to another particularly preferred embodiment, the composition according to the invention comprises at least one liquid fatty substance chosen from liquid hydrocarbons comprising more than 16 carbon atoms, in particular liquid petroleum jelly, liquid fatty alcohols, and mixtures thereof.

More preferentially, the composition according to the invention comprises at least one liquid fatty substance chosen from liquid hydrocarbons comprising more than 16 carbon atoms, in particular liquid petroleum jelly.

When the composition according to the invention comprises one or more fatty substances other than the hydrocarbons with a melting point of greater than 25° C., it comprises these fatty substances in a total content ranging from 10% to 60% by weight relative to the total weight of the composition.

When the composition according to the invention comprises one or more fatty substances other than the hydrocarbons with a melting point of greater than 25° C., it preferably comprises these fatty substances in a total content of greater than or equal to 12% by weight, preferentially greater than or equal to 15% by weight, better still greater than or equal to 20% by weight, even better still greater than or equal to 25% by weight, relative to the total weight of the composition.

When the composition according to the invention comprises one or more fatty substances other than the hydrocarbons with a melting point of greater than 25° C., it preferably comprises these fatty substances in a total content ranging from 10% to 60% by weight, preferably from 12% to 50% by weight, more preferentially from 15% to 40% by weight, better still from 20% to 35% by weight, even better still from 25% to 35% by weight, relative to the total weight of the composition.

When the composition according to the invention comprises one or more liquid fatty substances, it preferably comprises these liquid fatty substances in a total content ranging from 12% to 50% by weight, preferably from 15% to 40% by weight, more preferentially from 20% to 35% by weight, better still from 25% to 35% by weight, relative to the total weight of the composition.

### Surfactants

The composition according to the present invention can comprise one or more surfactants.

These surfactants may preferably be chosen from anionic surfactants, amphoteric surfactants, nonionic surfactants and cationic surfactants and/or mixtures thereof.

Preferably, the composition according to the invention comprises one or more surfactants.

“Anionic surfactant” means a surfactant comprising, as ionic or ionizable groups, only anionic groups. These anionic groups are preferably chosen from the groups CO₂H, CO₂⁻, SO₃H, SO₃⁻, OSO₃H, OSO₃⁻, H₂PO₃, HPO₃⁻, PO₃²⁻, H₂PO₂, HPO₂⁻, PO₂²⁻, POH and PO⁻.

As examples of anionic surfactants that may be used in the composition according to the invention, mention may be made of alkyl sulfates, alkyl ether sulfates, alkylamido ether sulfates, alkylaryl polyether sulfates, monoglyceride sulfates, alkyl sulfonates, alkylamide sulfonates, alkylaryl sulfonates, α-olefin sulfonates, paraffin sulfonates, alkyl sulfosuccinates, alkyl ether sulfosuccinates, alkylamide sulfosuccinates, alkyl sulfoacetates, acyl sarcosinates, acyl glutamates, alkyl sulfosuccinamates, acyl isethionates and N—(C1-C4)alkyl N-acyl taurates, salts of alkyl monoesters of polyglycoside-polycarboxylic acids, acyl lactylates, salts of D-galactoside uronic acids, salts of alkyl ether carboxylic acids, salts of alkylaryl ether carboxylic acids, salts of alkylamido ether carboxylic acids; and the corresponding non-salified forms of all these compounds; the alkyl and acyl groups of all these compounds (unless specified otherwise) generally comprising from 6 to 24 carbon atoms and the aryl group generally denoting a phenyl group.

Among the anionic surfactants, mention may also be made of salts of fatty acids, particularly C8-C24 fatty acids, preferably C12-C20 fatty acids.

These compounds may be oxyethylenated and then preferably comprise from 1 to 50 ethylene oxide units.

The salts of C₆-C₂₄ alkyl monoesters of polyglycoside-polycarboxylic acids may be chosen from C₆-C₂₄ alkyl polyglycoside-citrates, C₆-C₂₄ alkyl polyglycoside-tartrates and C₆-C₂₄ alkyl polyglycoside-sulfosuccinates.

When the anionic surfactant(s) are in salt form, they may be chosen from alkali metal salts such as the sodium or potassium salt and preferably the sodium salt, ammonium salts, amine salts and in particular amino alcohol salts or alkaline-earth metal salts such as the magnesium salt.

By way of example of amino alcohol salts, mention may particularly be made of monoethanolamine, diethanolamine and triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)aminomethane salts.

Alkali metal or alkaline-earth metal salts and in particular the sodium or magnesium salts are preferably used.

The anionic surfactants that are optionally present may be mild anionic surfactants, i.e. anionic surfactants without a sulfate function.

As regards mild anionic surfactants, mention may be made in particular of the following compounds and salts thereof, and also mixtures thereof: polyoxyalkylenated carboxylic acid alkyl ethers; polyoxyalkylenated carboxylic acid alkylaryl ethers; polyoxyalkylenated carboxylic acid alkylamido ethers, in particular those comprising 2 to 50 ethylene oxide groups; alkyl D-galactoside uronic acids; acylsarcosinates, acylglutamates; and alkylpolyglycoside carboxylic esters.

Use may be made most particularly of polyoxyalkylenated carboxylic acid alkyl ethers, for instance the carboxylic acid lauryl ether (4.5 EO) sold, for example, under the name AKYPO RLM 45 CA from KAO.

Among the anionic surfactants mentioned above, use is preferably made of the sulfated surfactants such as the alkyl sulfates or alkyl ether sulfates, and the acyl glutamates, salts of C12-C20 fatty acids, more preferentially alkyl sulfates and salts of C12-C20 fatty acids.

The amphoteric or zwitterionic surfactant(s) which can be used in the composition according to the invention are preferably nonsilicone surfactants and can particularly be optionally quaternized secondary or tertiary aliphatic amine derivatives, in which the aliphatic group is a linear or branched chain comprising from 8 to 22 carbon atoms, said amine derivatives containing at least one anionic group, for instance a carboxylate, sulfonate, sulfate, phosphate or phosphonate group.

Mention may in particular be made of (C₈-C₂₀)alkylbetaines, (C₈-C₂₀)alkylsulfobetaines, (C₈-C₂₀)alkylamido (C₁-C₆)alkylbetaines and (C₈-C₂₀)alkylamido (C₁-C₆)alkylsulfobetaines, and mixtures thereof.

Among the optionally quaternized secondary or tertiary aliphatic amine derivatives that may be used, as defined above, mention may also be made of the compounds having the respective structures (III) and (IV) below:

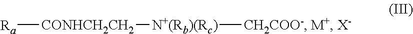

in which formula (III):
Rₐ represents a C₁₀ to C₃₀ alkyl or alkenyl group derived from an acid RₐCOOH which is preferably present in hydrolyzed coconut kernel oil; preferably, Rₐ represents a heptyl, nonyl or undecyl group;
R_{b} represents a β-hydroxyethyl group;
R_{c} represents a carboxymethyl group;
M⁺ represents a cationic counterion derived from an alkali metal or alkaline-earth metal, such as sodium, an ammonium ion or an ion derived from an organic amine; and
X⁻ represents an organic or inorganic anionic counterion, such as that chosen from halides, acetates, phosphates, nitrates, (C₁-C₄)alkyl sulfates, (C₁-C₄)alkyl- or (C₁-C₄)alkylaryl-sulfonates, in particular methyl sulfate and ethyl sulfate; or alternatively M⁺ and X⁻ are absent;

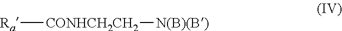

in which formula (IV):
B represents the group —CH₂CH₂OX′;
B′ represents the group —(CH₂)_{z}Y′, with z=1 or 2;
X′ represents the group —CH₂COOH, —CH₂—COOZ′, —CH₂CH₂COOH or CH₂CH₂—COOZ′, or a hydrogen atom;
Y′ represents the group —COOH, —COOZ′ or —CH₂CH(OH)SO₃H or the group CH₂CH(OH)SO₃—Z′;
Z′ represents a cationic counterion derived from an alkali metal or alkaline-earth metal, such as sodium, an ammonium ion or an ion derived from an organic amine;
Rₐ′ represents a C₁₀ to C₃₀ alkyl or alkenyl group of an acid Rₐ′—COOH which is preferably present in coconut kernel oil or in hydrolyzed linseed oil; preferably, Rₐ′ is an alkyl group, particularly a C₁₇ group, and its iso form, or an unsaturated C₁₇ group.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid and cocoamphodipropionic acid.

By way of example, mention may be made of the cocoamphodiacetate sold by Rhodia under the trade name Miranol® C2M Concentrate.

Use may also be made of compounds of formula (V):

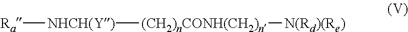

in which formula (V):
Y″ represents the group —COOH, —COOZ″ or —CH₂CH(OH)SO₃H or the group CH₂CH(OH)SO₃—Z″;
R_{d} and Rₑ, independently of each other, represent a C₁ to C₄ alkyl or hydroxyalkyl radical;
Z″ represents a cationic counterion derived from an alkali metal or alkaline-earth metal, such as sodium, an ammonium ion or an ion derived from an organic amine;
Rₐ″ represents a C₁₀ to C₃₀ alkyl or alkenyl group of an acid Rₐ″—COOH which is preferably present in coconut kernel oil or in hydrolyzed linseed oil; and
n and n′ denote, independently of each other, an integer ranging from 1 to 3.

Among the compounds of formula (V), mention may be made of the compound classified in the CTFA dictionary under the name sodium diethylaminopropyl cocoaspartamide and sold by the company Chimex under the name Chimexane HB.

These compounds may be used alone or as mixtures.

Among the amphoteric or zwitterionic surfactants mentioned above, use is advantageously made of (C₈-C₂₀)alkylbetaines, such as cocoyl betaine, (C₈-C₂₀)alkylamido (C₃-C₈)alkylbetaines, such as cocamidopropylbetaine, (C₈-C₂₀)alkylamphoacetates, (C₈-C₂₀)alkylamphodiacetates and mixtures thereof; and preferably (C₈-C₂₀)alkylbetaines, (C₈-C₂₀)alkylamido (C₃-C₈)alkylbetaines and mixtures thereof.

Preferentially, the amphoteric or zwitterionic surfactants are chosen from (C₈-C₂₀)alkylbetaines, (C₈-C₂₀)alkylamido (C₃-C₈)alkylbetaines, and mixtures thereof,

The nonionic surfactant(s) which can be used in the composition of the present invention are particularly described, for example, in the Handbook of Surfactants by M. R. Porter, published by Blackie & Son (Glasgow and London), 1991, pp. 116-178.

Mention may be made, as examples of nonionic surfactants, of the following compounds, alone or as a mixture:
oxyalkylenated (C₈-C₂₄) alkylphenols;
saturated or unsaturated, linear or branched, oxyalkylenated or glycerolated C₈-C₄₀ alcohols, preferably comprising one or two fatty chains;
saturated or unsaturated, linear or branched, oxyalkylenated C₈ to C₃₀ fatty acid amides;
esters of saturated or unsaturated, linear or branched, C₈ to C₃₀ acids and of polyethylene glycols;
fatty acid esters of sucrose;
preferably oxyethylenated esters of saturated or unsaturated, linear or branched, C₈ to C₃₀ acids and of sorbitol;
C₈-C₃₀ fatty acid esters of sorbitan;
polyoxyethylenated C₈-C₃₀ fatty acid esters of sorbitan;
(C₈-C₃₀)alkyl(poly) glucosides, (C₈-C₃₀)alkenyl(poly) glucosides, which are optionally oxyalkylenated (0 to 10 oxyalkylene units) and comprising from 1 to 15 glucose units, (C₈-C₃₀)alkyl(poly) glucoside esters;
saturated or unsaturated oxyethylenated plant oils;
condensates of ethylene oxide and/or of propylene oxide;
N—(C₈-C₃₀)alkylglucamine and N—(C₈-C₃₀) acylmethylglucamine derivatives;
amine oxides.

They are particularly chosen from alcohols, α-diols and (C₁-C₂₀) alkylphenols, these compounds being ethoxylated, propoxylated or glycerolated and having at least one fatty chain comprising, for example, from 8 to 24 carbon atoms and preferably from 8 to 18 carbon atoms, it being possible for the number of ethylene oxide or propylene oxide groups to range particularly from 1 to 200 and the number of glycerol groups to range particularly from 1 to 30.

Mention may also be made of condensates of ethylene oxide and of propylene oxide with fatty alcohols, ethoxylated fatty amides preferably having from 1 to 30 ethylene oxide units, polyglycerolated fatty amides comprising on average from 1 to 5, and in particular from 1.5 to 4, glycerol groups, fatty acid esters of sucrose, fatty acid esters of polyethylene glycol, oxyethylenated plant oils, N—(C₆-C₂₄ alkyl) glucamine derivatives, amine oxides such as (C10-C14 alkyl)amine oxides or N—(C₁₀-C₁₄ acyl)aminopropylmorpholine oxides.

The C₈-C₃₀ and preferably C₁₂-C₂₂ fatty acid esters (partiularly monoesters, diesters and triesters) of sorbitan may be chosen from:

Sorbitan Caprylate; Sorbitan Cocoate; Sorbitan Isostearate; Sorbitan Laurate; Sorbitan Oleate; Sorbitan Palmitate; Sorbitan Stearate; Sorbitan Diisostearate; Sorbitan Dioleate; Sorbitan Distearate; Sorbitan Sesquicaprylate; Sorbitan Sesquiisostearate; Sorbitan Sesquioleate; Sorbitan Sesquistearate; Sorbitan Triisostearate; Sorbitan Trioleate; Sorbitan Tristearate.

The esters (particularly monoesters, diesters, triesters) of C₈-C₃₀ fatty acids and of polyoxyethylenated sorbitan are preferably chosen from C₈-C₃₀ fatty acid ester(s) of oxyethylenated sorbitan having from 1 to 30 ethylene oxide units, preferably from 2 to 20 ethylene oxide units, more preferably still from 2 to 10 ethylene oxide units.

Preferentially, the C₈-C₃₀ fatty acid ester(s) of oxyethylenated sorbitan is (are) chosen from esters of C₁₂-C₁₈ fatty acids and of oxyethylenated sorbitan, in particular from oxyethylenated esters of lauric acid, of myristic acid, of cetylic acid and of stearic acid and of sorbitan.

Preferably, the C₈-C₃₀ fatty acid ester(s) of oxyethylenated sorbitan is (are) chosen from oxyethylenated (4 EO) sorbitan monolaurate (Polysorbate-21), oxyethylenated (20 EO) sorbitan monolaurate (Polysorbate-20), oxyethylenated (20 EO) sorbitan monopalmitate (Polysorbate-40), oxyethylenated (20 EO) sorbitan monostearate (Polysorbate-60), oxyethylenated (4 EO) sorbitan monostearate (Polysorbate-61), oxyethylenated (20 EO) sorbitan monooleate (Polysorbate-80), oxyethylenated (5 EO) sorbitan monooleate (Polysorbate-81), oxyethylenated (20 EO) sorbitan tristearate (Polysorbate-65), oxyethylenated (20 EO) sorbitan trioleate (Polysorbate-85).

The nonionic surfactant(s) are preferably chosen from ethoxylated C₈-C₂₄ fatty alcohols comprising from 1 to 200 ethylene oxide groups, preferably from 1 to 50 ethylene oxide groups, (C₆-C₂₄ alkyl) polyglycosides, esters of saturated or unsaturated, linear or branched, C₈-C₃₀ fatty acids and of glycerol, esters of C₈-C₃₀ fatty acids and of oxyethylenated sorbitan, and mixtures thereof, preferentially from ethoxylated C₈-C₂₄ fatty alcohols comprising from 1 to 50 ethylene oxide groups, (C₆-C₂₄ alkyl) polyglycosides, esters of saturated or unsaturated, linear or branched, C8-C30 fatty acids and of glycerol.

More preferentially, the nonionic surfactant(s) are chosen from ethoxylated C₈-C₂₄ fatty alcohols comprising from 1 to 200 ethylene oxide groups, preferably from 1 to 50 ethylene oxide groups.

The cationic surfactant(s) that may be used in the composition according to the invention are generally chosen from optionally polyoxyalkylenated primary, secondary or tertiary fatty amines, quaternary ammonium salts, and mixtures thereof.

The fatty amines generally comprise at least one C₈-C₃₀ hydrocarbon-based chain. Among the fatty amines that may be used according to the invention, mention may for example be made of stearylamidopropyldimethylamine and distearylamine.

As examples of quaternary ammonium salts, mention may particularly be made of:
those corresponding to general formula (VI) below:

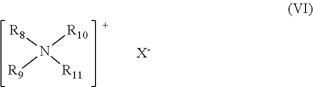

in which the groups R₈ to R₁₁, which may be identical or different, represent a linear or branched aliphatic group comprising from 1 to 30 carbon atoms, or an aromatic group such as aryl or alkylaryl, at least one of the groups R₈ to R₁₁ comprising from 8 to 30 carbon atoms and preferably from 12 to 24 carbon atoms. The aliphatic groups may comprise heteroatoms, particularly such as oxygen, nitrogen, sulfur and halogens.

The aliphatic groups are chosen, for example, from C1-C30 alkyl, C₁-C₃₀ alkoxy, polyoxy (C₂-C₆)alkylene, C₁-C₃₀ alkylamide, (C₁₂-C₂₂)alkylamido (C₂-C₆)alkyl, (C₁₂-C₂₂)alkyl acetate and C₁-C₃₀ hydroxyalkyl groups; X⁻ is an anion chosen from the group of halides, phosphates, acetates, lactates, (C₁-C₄)alkyl sulfates and (C₁-C₄)alkylsulfonates or (C₁-C₄)alkylarylsulfonates.

Among the quaternary ammonium salts of formula (VI), preference is given, firstly, to tetraalkylammonium chlorides, for example dialkyldimethylammonium or alkyltrimethylammonium chlorides in which the alkyl group comprises approximately 12 to 22 carbon atoms, in particular behenyltrimethylammonium chloride, distearyldimethylammonium chloride, cetyltrimethylammonium chloride or benzyldimethylstearylammonium chloride, or, secondly, to distearoylethylhydroxyethylmethylammonium methosulfate, dipalmitoylethylhydroxyethylammonium methosulfate or distearoylethylhydroxyethylammonium methosulfate, or also, finally, to palmitylamidopropyltrimethylammonium chloride or stearamidopropyldimethyl(myristyl acetate) ammonium chloride, sold under the name Ceraphyl® 70 by the company Van Dyk.
quaternary ammonium salts of imidazoline, for instance those of formula (VII) below:

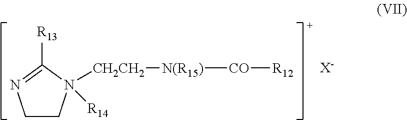

in which R12 represents an alkenyl or alkyl group comprising from 8 to 30 carbon atoms, for example tallow fatty acid derivatives, R13 represents a hydrogen atom, a C₁-C₄ alkyl group or an alkenyl or alkyl group comprising from 8 to 30 carbon atoms, R14 represents a C₁-C₄ alkyl group, R15 represents a hydrogen atom or a C₁-C₄ alkyl group, and X is an anion chosen from the group of halides, phosphates, acetates, lactates, (C₁-C₄)alkyl sulfates, and (C₁-C₄)alkylsulfonates or (C₁-C₄)alkylarylsulfonates.

Preferably, R12 and R13 denote a mixture of alkenyl or alkyl groups comprising from 12 to 21 carbon atoms, for example tallow fatty acid derivatives, R14 denotes a methyl group and R15 denotes a hydrogen atom. Such a product is sold, for example, under the name Rewoquat® W 75 by the company Rewo.
quaternary diammonium or triammonium salts, in particular of formula (VIII) below:

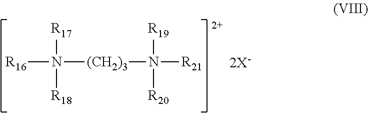

in which R16 denotes an alkyl group comprising approximately from 16 to 30 carbon atoms, which is optionally hydroxylated and/or interrupted with one or more oxygen atoms; R17 is chosen from hydrogen, an alkyl group comprising from 1 to 4 carbon atoms or a group —(CH2) 3-N+(R16a) (R17a) (R18a), R16a, R17a, R18a, R18, R19, R20 and R21, which are identical or different, are chosen from hydrogen or an alkyl group comprising from 1 to 4 carbon atoms, and X is an anion chosen from the group of halides, acetates, phosphates, nitrates, (C1-C4)alkyl sulfates, (C1-C4)alkylsulfonates or (C1-C4)alkylarylsulfonates, in particular methyl sulfate and ethyl sulfate.

Such compounds are, for example, Finquat CT-P, sold by the company Finetex (Quaternium 89), and Finquat CT, sold by the company Finetex (Quaternium 75).
quaternary ammonium salts containing one or more ester functions, for instance those of formula (IX) below:

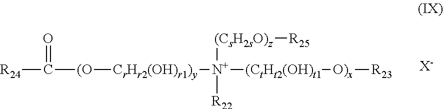

in which: R22 is chosen from C1-C6 alkyl groups and C1-C6 hydroxyalkyl or dihydroxyalkyl groups; R23 is chosen from: the group —C(O)R26, linear or branched, saturated or unsaturated C1-C22 hydrocarbon-based groups R27, or a hydrogen atom; R25 is chosen from: the group —C(O)R28, linear or branched, saturated or unsaturated C1-C6 hydrocarbon-based groups R29, or a hydrogen atom; R24, R26 and R28, which are identical or different, are chosen from linear or branched, saturated or unsaturated C7-C21 hydrocarbon-based groups; r, s and t, which are identical or different, are integers from 2 to 6; r1 and t1, which are identical or different, are 0 or 1; r2+r1=2 r and t1+12=2 t, y is an integer from 1 to 10, x and z, which are identical or different, are integers from 0 to 10, X⁻ is an organic or inorganic simple or complex anion, with the proviso that the sum x+y+z is from 1 to 15, that when x is 0, R23 denotes R27 and that when z is 0, R25 denotes R29.

The alkyl groups R22 may be linear or branched, and more particularly linear.

Preferably, R22 denotes a methyl, ethyl, hydroxyethyl or dihydroxypropyl group, and more particularly a methyl or ethyl group.

Advantageously, the sum x+y+z is from 1 to 10.

When R23 is a hydrocarbon-based group R27, it may be long and may have from 12 to 22 carbon atoms, or may be short and may have from 1 to 3 carbon atoms.

When R25 is a hydrocarbon-based group R29, it preferably has 1 to 3 carbon atoms.

Advantageously, R24, R26 and R28, which are identical or different, are chosen from linear or branched, saturated or unsaturated C11-C21 hydrocarbon-based groups, and more particularly from linear or branched, saturated or unsaturated C11-C21 alkyl and alkenyl groups.

Preferably, x and z, which are identical or different, are equal to 0 or 1.

Advantageously, y is equal to 1.

Preferably, r, s and t, which are identical or different, are equal to 2 or 3, and even more particularly are equal to 2.

The anion X⁻ is preferably a halide, preferably chloride, bromide or iodide, a (C1-C4)alkyl sulfate or a (C1-C4)alkyl- or (C1-C4)alkylaryl-sulfonate. However, use may be made of methanesulfonate, phosphate, nitrate, tosylate, an anion derived from an organic acid, such as acetate or lactate, or any other anion that is compatible with the ammonium bearing an ester function.

The anion X⁻ is even more particularly chloride, methyl sulfate or ethyl sulfate.

Use is made more particularly, in the composition according to the invention, of the ammonium salts of formula (XIII) in which: R22 denotes a methyl or ethyl group, x and y are equal to 1, z is equal to 0 or 1, r, s and t are equal to 2; R23 is chosen from: the group —C(O)R26, methyl, ethyl or C14-C22 hydrocarbon-based groups, or a hydrogen atom, R25 is chosen from: the group —C(O)R28, or a hydrogen atom, R24, R26 and R28, which are identical or different, are chosen from linear or branched, saturated or unsaturated C13-C17 hydrocarbon-based groups, and preferably from linear or branched, saturated or unsaturated C13-C17 alkyl and alkenyl groups.

Advantageously, the hydrocarbon-based groups are linear.

Among the compounds of formula (XIII), mention may for example be made of the salts, particularly the chloride or methyl sulfate, of diacyloxyethyldimethylammonium, diacyloxyethylhydroxyethylmethylammonium, monoacyloxyethyldihydroxyethylmethylammonium, triacyloxyethylmethylammonium or monoacyloxyethylhydroxyethyldimethylammonium, and mixtures thereof. The acyl groups preferably have 14 to 18 carbon atoms and originate more particularly from a plant oil such as palm oil or sunflower oil. When the compound contains several acyl groups, these groups may be identical or different.

These products are obtained, for example, by direct esterification of triethanolamine, triisopropanolamine, an alkyldiethanolamine or an alkyldiisopropanolamine, which are optionally oxyalkylenated, with fatty acids or with fatty acid mixtures of plant or animal origin, or by transesterification of the methyl esters thereof. This esterification is followed by a quaternization by means of an alkylating agent such as an alkyl halide, preferably methyl or ethyl halide, a dialkyl sulfate, preferably dimethyl or diethyl sulfate, methyl methanesulfonate, methyl para-toluenesulfonate, glycol chlorohydrin or glycerol chlorohydrin.

Such compounds are sold, for example, under the names Dehyquart® by the company Henkel, Stepanquat® by the company Stepan, Noxamium® by the company CECA or Rewoquat® WE 18 by the company Rewo-Witco.

The composition according to the invention may contain, for example, a mixture of quaternary ammonium monoester, diester and triester salts with a weight majority of diester salts.

Use may also be made of the ammonium salts containing at least one ester function that are described in U.S. Pat. Nos. 4,874,554 and 4,137,180.

Use may also be made of the behenoylhydroxypropyltrimethylammonium chloride sold, for example, by Kao under the name Quartamin BTC 131.

Preferably, the ammonium salts containing at least one ester function contain two ester functions.

Among the cationic surfactants, preference is more particularly given to choosing cetyltrimethylammonium, behenyltrimethylammonium and dipalmitoylethylhydroxyethyl-methylammonium salts, and mixtures thereof, and more particularly behenyl-trimethylammonium chloride, cetyltrimethylammonium chloride, and dipalmitoylethyl-hydroxyethylammonium methosulfate, and mixtures thereof.

Preferably, the surfactant(s) are chosen from anionic surfactants and nonionic surfactants, and mixtures thereof, more preferentially from anionic surfactants.

More preferentially, the surfactant(s) are chosen from the ethoxylated C₈-C₂₄ fatty alcohols comprising from 1 to 200 ethylene oxide groups, preferably from 1 to 50 ethylene oxide groups, alkyl sulfates, salts of C12-C20 fatty acids, and mixtures thereof, better still from alkyl sulfates, salts of C12-C20 fatty acids, and mixtures thereof.

According to one preferred embodiment, the composition comprises one or more surfactants chosen from the alkyl sulfates.

When the composition comprises one or more surfactant(s), the total content of surfactant(s) in the composition preferably ranges from 0.01% to 20% by weight, more preferentially from 0.1% to 15% by weight, better still from 0.5% to 10% by weight, even better still from 1% to 8% by weight, relative to the total weight of the composition.

When the composition comprises one or more nonionic and/or anionic surfactant(s), the total content of nonionic and/or anionic surfactant(s) in the composition preferably ranges from 0.01% to 20% by weight, more preferentially from 0.1% to 15% by weight, better still from 0.5% to 10% by weight, even better still from 1% to 8% by weight, relative to the total weight of the composition.

### Sequestrant

The composition according to the invention may comprise one or more sequestrant(s) (or chelating agents).

Preferably, the composition according to the invention comprises one or more sequestrant(s).

The definition of a “sequestrant” (or “chelating agent”) is well known to those skilled in the art and refers to a compound or a mixture of compounds that are able to form a chelate with a metal ion. A chelate is an inorganic complex in which a compound (the sequestrant or chelating agent) is coordinated to a metal ion, i.e. it forms one or more bonds with the metal ion (formation of a ring including the metal ion).

A sequestrant (or chelating agent) generally comprises at least two electron-donating atoms which enable the formation of bonds with the metal ion.

In the context of the present invention, the sequestrant(s) may be chosen from carboxylic acids, preferably aminocarboxylic acids, phosphonic acids, preferably aminophosphonic acids, polyphosphoric acids, preferably linear polyphosphoric acids, salts thereof, and derivatives thereof.

The salts are particularly alkali metal, alkaline-earth metal, ammonium and substituted ammonium salts.

By way of example of sequestrants based on carboxylic acids, mention may be made of the following compounds: diethylenetriaminepentaacetic acid (DTPA), ethylenediaminedisuccinic acid (EDDS) and trisodium ethylenediamine disuccinate such as Octaquest E30 from Octel, ethylenediaminetetraacetic acid (EDTA) and salts thereof such as disodium EDTA, tetrasodium EDTA, ethylenediamine-N,N′-diglutaric acid (EDDG), glycinamide-N,N′-disuccinic acid (GADS), 2-hydroxypropylenediamine-N, N′-disuccinic acid (HPDDS), ethylenediamine-N,N′-bis(ortho-hydroxyphenylacetic acid) (EDDHA), N,N′-bis(2-hydroxybenzyl)ethylenediamine-N,N′-diacetic acid (HBED), nitrilotriacetic acid (NTA), methylglycinediacetic acid (MGDA), N-2-hydroxyethyl-N,N-diacetic acid and glyceryliminodiacetic acid (as described in documents EP-A-317 542 and EP-A-399 133), iminodiacetic acid-N-2-hydroxypropylsulfonic acid and aspartic acid-N-carboxymethyl-N-2-hydroxypropyl-3-sulfonic acid (as described in EP-A-516 102), beta-alanine-N, N′-diacetic acid, aspartic acid-N,N′-diacetic acid, and aspartic acid-N-monoacetic acid (described in EP-A-509 382), chelating agents based on iminodisuccinic acid (IDSA) (as described in EP-A-509 382), ethanoldiglycine acid, phosphonobutane tricarboxylic acid such as the compound sold by Bayer under the reference Bayhibit AM, N,N-dicarboxymethylglutamic acid and salts thereof such as tetrasodium glutamate diacetate (GLDA) such as Dissolvine GL38 or 45S from Akzo Nobel.

By way of example of chelating agents based on mono- or polyphosphonic acid, mention may be made of the following compounds: diethylenetriamine-penta (methylene phosphonic acid) (DTPMP), ethane-1-hydroxy-1,1,2-triphosphonic acid (E1HTP), ethane-2-hydroxy-1,1,2-triphosphonic acid (E2HTP), ethane-1-hydroxy-1,1-triphosphonic acid (EHDP), ethane-1,1,2-triphosphonic acid (ETP), ethylenediaminetetramethylene phosphonic acid (EDTMP), hydroxyethane-1,1 diphosphonic acid (HEDP, or etidronic acid), and salts such as disodium etidronate, tetrasodium etidronate.

By way of example of chelating agents based on polyphosphoric acid, mention may be made of the following compounds: sodium tripolyphosphate (STP), tetrasodium diphosphate, hexametaphosphoric acid, sodium metaphosphate, phytic acid.

According to one embodiment, the sequestrant(s) useful according to the invention are phosphorus-based sequestrants, i.e. sequestrants which comprise one or more phosphorus atoms, preferably at least two phosphorus atoms.

The phosphorus-based sequestrant(s) used in the composition according to the invention are preferably chosen from:
inorganic phosphorus-based derivatives preferably chosen from alkali metal or alkaline earth metal, preferably alkali metal, phosphates and pyrophosphates, such as sodium pyrophosphate, potassium pyrophosphate, sodium pyrophosphate decahydrate; and alkali metal or alkaline earth metal, preferably alkali metal, polyphosphates, such as sodium hexametaphosphate, sodium polyphosphate, sodium tripolyphosphate, sodium trimetaphosphate; which are optionally hydrated, and mixtures thereof;
organic phosphorus-based derivatives, for instance organic (poly)phosphates and (poly)phosphonates, such as etidronic acid and/or alkali metal or alkaline-earth metal salts thereof, such as tetrasodium etidronate, disodium etidronate and mixtures thereof.

Preferably, the phosphorus-based sequestrant(s) is (are) chosen from linear or cyclic compounds comprising at least two phosphorus atoms bonded together covalently via at least one linker L comprising at least one oxygen atom and/or at least one carbon atom.

The phosphorus-based sequestrant(s) may be chosen from inorganic phosphorus-based derivatives, preferably comprising at least two phosphorus atoms. More preferentially, the phosphorus-based sequestrant(s) are chosen from alkali metal or alkaline-earth metal pyrophosphates, better still from alkali metal pyrophosphates, in particular sodium pyrophosphate (also known as tetrasodium pyrophosphate).

The phosphorus-based sequestrant(s) may be chosen from organic phosphorus-based derivatives, preferably comprising at least two phosphorus atoms. More preferentially, the phosphorus-based sequestrant(s) is (are) chosen from etidronic acid (also known as 1-hydroxyethane-1,1-diphosphonic acid) and/or alkali metal or alkaline-earth metal, preferably alkali metal, salts thereof, for instance tetrasodium etidronate and disodium etidronate.

Thus, preferably, the phosphorus-based sequestrant(s) are chosen from alkali metal pyrophosphates, etidronic acid and/or alkali metal salts thereof, and a mixture of these compounds.

Particularly preferably, the phosphorus-based sequestrant(s) are chosen from tetrasodium etidronate, disodium etidronate, etidronic acid, tetrasodium pyrophosphate, and a mixture of these compounds.

According to the present invention, the sequestrants are preferably chosen from diethylenetriaminepentaacetic acid (DTPA) and salts thereof, diethylenediaminetetraacetic acid (EDTA) and salts thereof, ethylenediaminedisuccinic acid (EDDS) and salts thereof, etidronic acid and salts thereof, N,N-dicarboxymethylglutamic acid and salts thereof, N,N-dicarboxymethylglutamic acid and salts thereof (GLDA), and mixtures thereof.

Among the salts of these compounds, preference is given to the alkali metal salts and in particular the sodium or potassium salts.

When the composition comprises one or more sequestrants, the total content of the sequestrant(s) preferably ranges from 0.001% to 15% by weight, more preferentially from 0.05% to 10% by weight, better still from 0.01% to 8% by weight, even better still from 0.05% to 5% by weight, relative to the total weight of the composition.

### Alkaline Agent

The composition according to the present invention may also comprise one or more mineral, organic or hybrid alkaline agent(s).

Preferably, the composition according to the invention comprises one or more alkaline agent(s).

For the purposes of the present invention, the terms “alkaline agent” and “basifying agent” are used without distinction.

The mineral basifying agent(s) are preferably chosen from aqueous ammonia, alkali metal carbonates or bicarbonates such as sodium (hydrogen) carbonate and potassium (hydrogen) carbonate, alkali metal or alkaline-earth metal phosphates such as sodium phosphates or potassium phosphates, sodium or potassium hydroxides, alkali metal or alkaline-earth metal silicates or metasilicates such as sodium metasilicate, and mixtures thereof.

The organic basifying agent(s) are preferably chosen from alkanolamines, organic amines other than alkanolamines, oxyethylenated and/or oxypropylenated ethylenediamines, 1,3-diaminopropane, spermine or spermidine and mixtures thereof.

“Alkanolamine” means an organic amine comprising a primary, secondary or tertiary amine function, and one or more linear or branched C1-C8 alkyl groups bearing one or more hydroxyl radicals.

Organic amines chosen from alkanolamines such as monoalkanolamines, dialkanolamines or trialkanolamines comprising one to three identical or different C1-C4 hydroxyalkyl radicals are in particular suitable for carrying out the invention.

In particular, the alkanolamine(s) are chosen from monoethanolamine (MEA), diethanolamine, triethanolamine, monoisopropanolamine, diisopropanolamine, N,N-dimethylethanolamine, 2-amino-2-methyl-1-propanol, triisopropanolamine, 2-amino-2-methyl-propane-1,3-diol, 3-amino-1,2-propanediol, 3-dimethylamino-1,2-propanediol, tris(hydroxymethyl)aminomethane and mixtures thereof.

The organic amine may also be chosen from organic amines of heterocyclic type. Besides histidine that has already been mentioned in the amino acids, mention may in particular be made of pyridine, piperidine, imidazole, triazole, tetrazole and benzimidazole. The organic amine may also be chosen from amino acid dipeptides. As amino acid dipeptides that may be used in the present invention, mention may particularly be made of carnosine, anserine and balenine. The organic amine may also be chosen from compounds comprising a guanidine function. As amines of this type other than arginine that may be used in the present invention, mention may particularly be made of creatine, creatinine, 1,1-dimethylguanidine, 1,1-diethylguanidine, glycocyamine, metformin, agmatine, n-amidoalanine, 3-guanidinopropionic acid, 4-guanidinobutyric acid and 2-([amino(imino)methyl]amino)ethane-1-sulfonic acid.

Use may be made in particular of guanidine carbonate or monoethanolamine hydrochloride as hybrid compounds.

The alkaline agent(s) that may be used according to the invention is (are) preferably chosen from alkanolamines such as monoethanolamine, diethanolamine, triethanolamine; aqueous ammonia, carbonates or bicarbonates such as sodium (hydrogen) carbonate and potassium (hydrogen) carbonate, alkali metal or alkaline-earth metal silicates or metasilicates such as sodium silicate and metasilicate and mixtures thereof, more preferentially from alkali metal or alkaline-earth metal silicates or metasilicates such as sodium silicate and metasilicate, and mixtures thereof.

In a particular embodiment, the composition according to the invention is free of aqueous ammonia.

When the composition comprises one or more alkaline agents, it comprises same in a total content ranging preferably from 0.1% to 50% by weight, more preferentially from 1% to 40% by weight, better still from 5% to 35% by weight, even better still from 10% to 30% by weight, relative to the total weight of the composition.

According to a particular embodiment, the composition according to the invention comprises at least one (meta) silicate. According to this embodiment, the total content of alkali metal or alkaline-earth metal silicate(s) or metasilicate(s), preferably of sodium metasilicate or silicate, preferably varies from 0.1% to 50% by weight, more preferentially from 1% to 40% by weight, better still from 5% to 35% by weight, even better still from 10% to 30% by weight, relative to the total weight of the composition.

### Solvent

The composition according to the invention may also comprise at least one organic solvent.

By way of organic solvent, mention may for example be made of linear or branched C2 to C4 alkanols, such as ethanol and isopropanol; polyols and polyol ethers such as glycerol, 2-butoxyethanol, propylene glycol, dipropylene glycol, propane-1,3-diol, propylene glycol monomethyl ether, and diethylene glycol monoethyl ether and monomethyl ether, and also aromatic alcohols or ethers, such as benzyl alcohol or phenoxyethanol, and mixtures thereof.

When the composition comprises one or more organic solvents, the total content of the organic solvent(s) preferably ranges from 0.01% to 20% by weight, preferably from 0.05% to 15% by weight and preferentially from 0.1% to 10% by weight relative to the total weight of the composition.

Preferably, the composition according to the invention is anhydrous. “Anhydrous composition” means a composition which does not comprise any, or only very little, water, particularly less than 0.5% water, especially less than 0.1%, even better still less than 0.05%, indeed even less than 0.01% water, relative to the total weight of the composition. In particular, the composition according to the invention does not comprise any water added during its preparation, it being possible for the water which may be present to be provided by the starting materials used for its preparation.

### Additives

The composition according to the invention can optionally comprise one or more additives, other than the compounds of the invention, and among which mention may be made of polymers, other than the associative polymers and non-associative polysaccharides, mineral thickening agents, antidandruff agents, antiseborrhoeic agents, agents for combating hair loss and/or for promoting hair growth, vitamins and provitamins including panthenol, sunscreens, mineral or organic pigments, plasticizers, solubilizers, opacifying or pearlescent agents, antioxidants, fragrances or preservatives.

Of course, those skilled in the art will take care to choose this or these optional additional compounds such that the advantageous properties intrinsically attached to the composition according to the invention are not, or not substantially, adversely affected by the envisaged addition(s).

The above additives may generally be present in an amount, for each of them, of between 0 and 20% by weight, relative to the total weight of the composition.

According to one embodiment, the composition which has just been described does not comprise hydrogen peroxide. This composition is intended to be mixed at the time of use with a composition comprising hydrogen peroxide.

Before mixing with a composition comprising hydrogen peroxide, the composition according to the invention is preferably in the form of a cream.

Preferably, the composition according to the invention, before mixing with a composition comprising hydrogen peroxide, has a viscosity of greater than or equal to 100 poises (100 Pa·s), preferably greater than or equal to 130 poises (130 Pa·s), more preferably still between 130 and 250 poises (130 and 250 Pa·s), measured at 25° C. and at a shear rate of 1s⁻¹; it being possible for this viscosity to be determined by means of a Thermo Haake RS600 rotational rheometer fitted with plate-plate geometry, Ø 35 mm with a 1 mm gap.

As a variant, the composition according to the invention may comprise hydrogen peroxide. When it comprises hydrogen peroxide, the composition is a ready-to-use composition.

According to a preferred embodiment, the ready-to-use composition comprises:
one or more peroxygenated salts;
one or more associative polymers;
one or more non-associative polysaccharides;
one or more hydrocarbon(s) with a melting point of greater than 25° C.; and
hydrogen peroxide.

According to a particular embodiment, the ready-to-use composition comprises:
one or more persulfate(s);
one or more associative polymers;
one or more non-associative polysaccharides;
one or more hydrocarbon(s) with a melting point of greater than 25° C.; and
hydrogen peroxide.

Preferably, the ready-to-use composition is also in the form of a cream.

Preferably, the pH of the ready-to-use composition ranges from 8 to 13, preferentially from 9 to 12.

### Process

The present invention also relates to a process for lightening keratin fibres, preferably human keratin fibres, particularly the hair, comprising a step of applying, to said keratin fibres, an effective amount of a composition as defined above.

Preferably, the composition applied to the keratin fibres results from the mixing, before use, of a composition according to the invention which does not comprise hydrogen peroxide with a composition comprising hydrogen peroxide.

According to a preferred embodiment, the process for lightening keratin fibres, preferably human keratin fibres, particularly the hair, according to the invention comprises:
(i) a step of mixing a composition A according to the invention, as described above and which does not comprise hydrogen peroxide, with a composition B comprising hydrogen peroxide,
(ii) a step of applying, to said keratin fibres, the composition resulting from the mixture obtained in step (i).

The composition resulting from the mixture of step (i) is referred to as a ready-to-use composition.

This ready-to-use composition may comprise one or more ingredients from those described above. This ready-to-use composition may also comprise water, particularly originating from the composition containing hydrogen peroxide.

The ready-to-use composition may be applied to wet or dry keratin fibres. After the treatment, the keratin fibres are optionally rinsed with water, optionally washed with a shampoo and then rinsed with water, before being dried or left to dry.

This mixing step is preferably performed at the time of use, just before the composition resulting from the mixing is applied to the hair.

Preferably, compositions A and B are mixed in an A/B weight ratio ranging from 0.1 to 2, preferentially from 0.3 to 1.5, better still from 0.5 to 1.

Preferably, the composition B comprises hydrogen peroxide in a content ranging from 0.1% to 50%, more particularly from 0.5% to 20%, and even more preferentially from 1% to 15% by weight, relative to the total weight of the composition B.

Composition B is preferably an aqueous composition. In particular, it comprises more than 10% by weight of water, preferably more than 30% by weight of water and more advantageously still more than 50% by weight of water.

It may also comprise one or more organic solvents chosen from those listed previously; these solvents more particularly representing, when they are present, from 0.1% to 30% by weight and preferably from 0.3% to 20% by weight, relative to the weight of the oxidizing composition.

Composition B also preferably comprises one or more acidifying agents. Among the acidifying agents, mention may be made, by way of example, of mineral or organic acids, such as hydrochloric acid, orthophosphoric acid, sulfuric acid, carboxylic acids, such as acetic acid, tartaric acid, citric acid or lactic acid, and sulfonic acids.

Composition B preferably comprises one or more fatty substances such as those described above, preferably chosen from fatty alcohols, liquid hydrocarbons comprising more than 16 carbon atoms and mixtures thereof.

Composition B may also comprise surfactants and thickening polymers.

The pH of composition B, if it is aqueous, is usually between less than 7, preferably between 1 and 5 and preferentially between 1.5 and 4.5.

### Kit

Another subject of the invention is a device having at least two compartments, for lightening keratin fibres, comprising at least one first compartment containing a composition A as described above, and at least one second compartment containing a composition B comprising hydrogen peroxide as described above.

The compositions of the device according to the invention are packaged in separate compartments, optionally accompanied by suitable application means, which are identical or different, such as fine brushes, coarse brushes or sponges.

The device mentioned above may also be equipped with a means for dispensing the desired mixture on the hair, for instance the devices described in patent FR 2 586 913.

Finally, the present invention relates to the use of a composition according to the invention as described above, for lightening keratin fibres, and in particular the hair.

The examples that follow serve to illustrate the invention without, however, being limiting in nature.

### EXAMPLES

In the examples which follow, all the amounts are shown as percentage by weight of active material (AM) relative to the total weight of the composition (unless otherwise indicated).

### Example 1

### Compositions A1 and A2

The composition A1 according to the invention and the comparative example A2 were prepared from the ingredients of which the contents are indicated in the table below:

TABLE 1

| | A1 | A2 |
|---|---|---|
| POTASSIUM PERSULFATE | 28.38 | 28.38 |
| AMMONIUM PERSULFATE | 9.95 | 9.95 |
| SODIUM SILICATE | 18 | 18 |
| SODIUM METASILICATE | 5 | 5 |
| SODIUM STEARATE | 4.72 | 4.72 |
| HYDROXYETHYLCELLULOSE | 0.75 | — |
| ACRYLATES/C10-30 ALKYL | 0.75 | 1.5 |
| ACRYLATE CROSSPOLYMER | | |
| SODIUM LAURYL SULFATE | 1 | 1 |
| DISODIUM EDTA | 0.5 | 0.5 |
| SILICA | 0.05 | 0.05 |
| MINERAL OIL/PARAFFINUM | 29 | 29 |
| LIQUIDUM | | |
| GLYCERYL STEARATE | 0.01 | 0.01 |
| POLYETHYLENE* | 1.89 | 1.89 |

*polyethylene having number-average molecular weight of 655, and having melting point of 96° C.

Composition B The composition B was prepared from the ingredients of which the contents are indicated in the table below:

TABLE 2

| | B |
|---|---|
| TETRASODIUM PYROPHOSPHATE | 0.04 |
| SODIUM SALICYLATE | 0.035 |
| ACRYLATES/BEHENETH-25 | 0.4 |
| METHACRYLATE COPOLYMER | |
| CETEARETH-33 | 2 |
| HYDROGEN PEROXIDE | 9 |
| CETEARYL ALCOHOL | 8 |
| TETRASODIUM ETIDRONATE | 0.06 |
| PHOSPHORIC ACID | qs pH 3 ± 0.2 |
| WATER/AQUA | qs 100 |

At the time of use, compositions A1 and A2 were respectively mixed with composition B at a weight ratio of 1+1.5 in order to obtain the mixtures M1 and M2.

The mixture M1 obtained is homogeneous, constituted of a single visible phase which is creamy whereas the mixture M2 is thicker, gelled and clumpy.

The viscosity of the mixtures M1 and M2 was also measured using a Rheomat RM 180 rheometer (200 rpm, 25° C., measured at 30 s, spindle 4).

The results obtained are the following (expressed in mPa·s):

TABLE 3

| | Mixture M1 | Mixture M2 |
|---|---|---|
| Viscosity | 7370 mPa · s | 9245 mPa · s |

Mixture M1 has a lower viscosity than mixture M2.

Thus, mixture M1 according to the invention is easy-to-use, i.e. easy to apply and spread uniformly on the hair whereas mixture M2 is more difficult to spread on the hair.

Furthermore, the stability of the mixtures M1 and M2 over time was studied by observing the change in their macroscopic appearance, at ambient temperature (25° C.), 24 hours after production of the mixtures.

After 24 hours, mixture M1 is still constituted of a single homogeneous phase whereas a phenomenon of syneresis is observed with mixture M2 which has two separate phases.

Composition A1 according to the invention results in a mixture M1 that is stable over time whereas comparative composition A2 results in a thicker mixture which is unstable.

Mixture M1 also makes it possible to obtain a uniform bleaching of the hair.

### Example 2

### Compositions A3, A4

The composition A3 according to the invention and the comparative composition A4 were prepared from the ingredients of which the contents are indicated in the table below:

TABLE 4

| | A3 | A4 |
|---|---|---|
| POTASSIUM PERSULFATE | 24.48 | 24.48 |
| AMMONIUM PERSULFATE | 9.95 | 9.95 |
| SODIUM SILICATE | 18 | 18 |
| SODIUM METASILICATE | 5 | 5 |
| SODIUM STEARATE | 4.72 | 4.72 |
| HYDROXYETHYLCELLULOSE | 0.75 | 0.75 |
| ACRYLATES/C10-30 ALKYL ACRYLATE | 0.75 | |
| CROSSPOLYMER | | |
| CARBOMER | | 0.75 |
| SODIUM LAURYL SULFATE | 1 | 1 |
| DISODIUM EDTA | 0.5 | 0.5 |
| SILICA | 0.05 | 0.05 |
| MINERAL OIL/PARAFFINUM LIQUIDUM | 29 | 29 |
| GLYCERYL STEARATE | 0.01 | 0.01 |
| POLYETHYLENE* | 1.89 | 1.89 |
| GLYCINE | 0.6 | 0.6 |
| CITRIC ACID | 3.33 | 3.33 |

*polyethylene having number-average molecular weight of 655, and melting point of 96° C.

### Composition B′

Composition B′ was prepared from the ingredients of which the contents are indicated in the table below:

TABLE 5

| | B′ |
|---|---|
| TETRASODIUM PYROPHOSPHATE | 0.04 |
| SODIUM SALICYLATE | 0.035 |
| TRIDECETH-2 CARBOXAMIDE MEA | 0.82 |
| CETEARETH-25 | 0.57 |
| GLYCERIN | 0.5 |
| HYDROGEN PEROXIDE | 9 |
| CETEARYL ALCOHOL | 2.25 |
| TETRASODIUM ETIDRONATE | 0.06 |
| PHOSPHORIC ACID | to pH 2.2 +/− 0.2 |
| WATER/AQUA | Up to 100 |

At the time of use, compositions A3 and A4 were respectively mixed with composition B′ at a weight ratio of 1+1.5 in order to obtain the mixtures M3 and M4.

Each of mixtures M3 and M4 was then applied to locks of natural chestnut-brown hair (tone depth 4) at an amount of 10 g of mixture for 1 g of lock of hair.

After a leave-on time of 50 minutes at 33° C., the locks were rinsed, then washed and dried at 60° C. in an oven.

The lightening of the hair was evaluated in the L*a*b* system, using a Konica Minolta CM-3600A spectrocolorimeter (illuminant D65, angle 10°, specular component included) in the CIELab system.

In this system, L* represents the lightness, a* represents the green/red colour axis and b* represents the blue/yellow colour axis.

The level of lightening is represented by the colour difference ΔE between the locks of natural hair before treatment and the lightened locks of hair, ΔE being obtained from the formula:

$$Δ E = \sqrt{{( {L}^{⋆} - {L}_{o}^{⋆} )}^{2} + {( {a}^{⋆} - {a}_{o}^{⋆} )}^{2} + {( {b}^{⋆} - {b}_{o}^{⋆} )}^{2}}$$

in which L* represents the intensity and a* and b* represent the chromaticity of the natural hair, and L₀* represents the intensity and a₀* and b₀* represent the chromaticity of the lightened hair.

The greater the value of ΔE, the more the hair has been lightened.

### Results

TABLE 6

| | L | a | b | ΔE |
|---|---|---|---|---|
| Control before treatment | 21.39 | 2.72 | 2.94 | — |
| M3 | 48.21 | 12.02 | 31.33 | 40.15 |
| M4 | 45.69 | 11.97 | 29 | 36.81 |

Mixture M3 leads to better lightening of the hair compared to mixture M4.

1-20. (canceled)

21. A composition comprising:
(i) one or more peroxygenated salts;
(ii) one or more associative polymers;
(iii) one or more non-associative polysaccharides; and
(iv) one or more hydrocarbons with a melting point of greater than 25° C.

22. The composition of claim 21, wherein the one or more peroxygenated salts (i) is chosen from alkali metal persulfates, alkaline-earth metal persulfates, ammonium persulfates, or mixtures of two or more thereof.

23. The composition of claim 21, wherein the one or more peroxygenated salts (i) is chosen from (bis)tetrabutylammonium persulfate, barium persulfate, magnesium persulfate, calcium persulfate, sodium persulfate, potassium persulfate, ammonium persulfate, or mixtures of two or more thereof.

24. The composition of claim 21, wherein the total amount of peroxygenated salt(s) (i) ranges from 1% to 60% by weight, relative to the total weight of the composition.

25. The composition of claim 21, wherein one or more hydrocarbons with a melting point of greater than 25° C. (iv) is chosen from microcrystalline waxes, polyethylene waxes, Fischer-Tropsch waxes, paraffin waxes, ozokerite, or mixtures of two or more thereof.

26. The composition of claim 21, wherein one or more hydrocarbons with a melting point of greater than 25° C. (iv) is chosen from ethylene homopolymers with a melting point greater than or equal to 30° C.

27. The composition of claim 21, wherein the total amount of hydrocarbon(s) with a melting point of greater than 25° C. (iv) ranges from 0.1% to 30% by weight, relative to the total weight of the composition.

28. The composition of claim 21, wherein the one or more associative polymer(s) (ii) is chosen from anionic associative polymers.

29. The composition of claim 21, wherein the one or more associative polymer(s) (ii) is chosen from acrylic acid homopolymers or copolymers, methacrylic acid homopolymers or copolymers, or mixtures thereof.

30. The composition of claim 21, wherein the one or more associative polymer(s) (ii) is chosen from:
(a) polymers comprising at least one hydrophilic unit and at least one fatty-chain allyl ether unit;
(b) polymers comprising at least one hydrophilic unit of unsaturated olefinic carboxylic acid type, and at least one hydrophobic unit of a (C10-C30)alkyl ester of an unsaturated carboxylic acid;
(c) maleic anhydride/C30-C38 α-olefin/alkyl maleate terpolymers;
(d) acrylic terpolymers comprising:
from about 20% to about 70% by weight of an α,β-monoethylenically unsaturated carboxylic acid [A],
from about 20% to about 80% by weight of an α,β-monoethylenically unsaturated non-surfactant monomer other than [A],
from about 0.5% to about 60% by weight of a nonionic monourethane;
(e) copolymers comprising monomers chosen from an α,β-monoethylenically unsaturated carboxylic acid, an ester of an α,β-monoethylenically unsaturated carboxylic acid, an ester an oxyalkylenated fatty alcohol, or mixtures of monomers thereof;
(f) amphiphilic polymers comprising at least one ethylenically unsaturated monomer comprising a sulfonic group in free or partially or totally neutralized form, or mixtures thereof, wherein the sulfonic group comprises at least one hydrophobic part; or mixtures of two or more thereof.

31. The composition of claim 21, wherein the total amount of the associative polymer(s) (ii) ranges from 0.01% to 15% by weight, relative to the total weight of the composition.

32. The composition of claim 21, wherein the one or more non-associative polysaccharide(s) (iii) is chosen from polymers of (g)-(k), derivatives thereof, or mixtures of two or more thereof:
g) tree or shrub exudates, comprising:
gum arabic;
ghatti gum;
karaya gum;
gum tragacanth;
h) gums derived from algae, comprising:
agar;
alginates;
carrageenans and furcellerans;
i) gums derived from seeds or tubers, comprising:
guar gum;
locust bean gum;
fenugreek gum;
tamarind gum;
konjac gum;
j) microbial gums, comprising:
xanthan gum;
gellan gum;
scleroglucan gum;
k) polymers extracted from plants, comprising:
celluloses;
starches;
inulin.

33. The composition of claim 21, wherein the one or more non-associative polysaccharides (iii) is chosen from cellulose ethers, cellulose esters, or mixtures thereof.

34. The composition of claim 21, wherein the total amount of non-associative polysaccharide(s) (iii) ranges from 0.01% to 15% by weight, relative to the total weight of the composition.

35. The composition of claim 21, further comprising (v) one or more alkaline agents, wherein the total amount of alkaline agent(s) (v) ranges from 10% to 30% by weight, relative to the total weight of the composition.

36. The composition of claim 35, wherein the one or more alkaline agents (v) is chosen from aqueous ammonia, alkali metal carbonates, alkali metal bicarbonates, alkali metal phosphates, alkaline-earth metal phosphates, sodium hydroxides, potassium hydroxides, alkali metal silicates, alkaline-earth metal silicates, alkali metal metasilicates, alkaline-earth metal metasilicates, or mixtures of two or more thereof.

37. The composition of claim 21, wherein the composition does not comprise hydrogen peroxide.

38. The composition of claim 21, further comprising hydrogen peroxide.

39. A method for lightening keratin fibers, comprising:
(1) mixing a composition A with a composition B comprising hydrogen peroxide, wherein composition A comprises:
(i) one or more peroxygenated salts;
(ii) one or more associative polymers;
(iii) one or more non-associative polysaccharides; and
(iv) one or more hydrocarbons with a melting point of greater than 25° C.; and
(2) applying the mixture to the keratin fibers.

40. A device for lightening keratin fibers comprising at least two compartments,
wherein the first compartment comprises a composition A comprising:
(i) one or more peroxygenated salts;
(ii) one or more associative polymers;
(iii) one or more non-associative polysaccharides; and
(iv) one or more hydrocarbons with a melting point of greater than 25° C.;
wherein the second compartment comprises a composition B comprising hydrogen peroxide.
